(19) United States
(12) Patent Application Publication
(10) Pub. No.: US 2024/0066185 A1
(43) Pub. Date: Feb. 29, 2024

(54) MULTILAYERED VASCULARIZED CONSTRUCT

(71) Applicant: University of Washington, Seattle, WA (US)

(72) Inventors: Ying Zheng, Seattle, WA (US); Nicole Zeinstra, Seattle, WA (US); Ariana Frey, Seattle, WA (US)

(73) Assignee: University of Washington, Seattle, WA (US)

(21) Appl. No.: 18/458,120

(22) Filed: Aug. 29, 2023

Related U.S. Application Data
(60) Provisional application No. 63/374,202, filed on Aug. 31, 2022.

(51) Int. Cl. A61L 27/38; A61L 27/24; C12N 5/071; C12N 5/077

(52) U.S. Cl. CPC A61L 27/3808; A61L 27/24; A61L 27/3804; A61L 27/3826; C12N 5/0658; C12N 5/069; C12N 2533/54

(57) ABSTRACT

Compositions and methods for modular fabrication of large, three-dimensional, perfusable networks of vascularized tissue are provided. Each layer within the tissue may include the same or different cell types and have the same or different types of vasculature. In some aspects layers of tissue together form the large, three-dimensional perfusable networks as each layer contains a portion of the larger tissue and the combination of the various layers creates a tertiary structure of integrated perfusable networks that exhibit remodeling and evenly distributed perfusion among layers while maintaining a patterned open-lumen architecture.

### CROSS-REFERENCE TO RELATED APPLICATION

This application claims the priority of U.S. Provisional Patent Application No. 63/374,202, filed on Aug. 31, 2022. U.S. Provisional Patent Application No. 63/374,202 is incorporated by reference herein in its entirety.

### STATEMENT REGARDING FEDERALLY SPONSORED RESEARCH OR DEVELOPMENT

This invention was made with government support under Grant No. R01HL141570, Grant No. T32EB032787-01, Grant No. F31HL158061, and Grant No. T32EB032787, each of which was awarded by the National Institutes of Health. The government has certain rights in the invention.

### BACKGROUND

Tissue engineering holds promise in generating biomimetic tissue for regenerative medicine, disease modeling, and drug development (Mandrycky, C., Phong, K. & Zheng, Y. MRS Commun 7, 332-347 (2017)). Fabrication of large-scale engineered tissues can involve extensive vascularization to support tissue survival and function. Early work on vascularization in engineered tissues utilized endothelial cells to form self-assembled vascular networks (Gao, L. et al. Circulation 137, 1712-1730 (2018); Stevens, K. R. et al. Proceedings of the NationalAcademy of Sciences of the United States of America 106, 16568-73 (2009); Tulloch, N. L. et al. Circulation research 109, 47-59 (2011); Sekine, H. et al. Circulation 118, S145-52 (2008)). However, while the addition of endothelial cells improved maturation and tissue function, tissue prefusion was limited, leading to tissues with a thickness of <500 μm and poor integration with host vasculature (Coulombe, K. L. K. & Murry, C. E. IEEE Northeast Bioengineering Conference 2014, (2014)).

In an experiment by Sekine et al., cell sheets containing endothelial cells were stacked three at a time onto a vascular bed and became perfusable after vascular integration (Sekine, H. et al. Nature communications 4, 1399 (2013)). However, this method required days to establish perfusion from the host and the layers themselves were not perfusable at the time of fabrication. In another study, Zhang et al. stacked perfusable biodegradable scaffolds that were generated via lithography (Zhang, B. et al. Nature materials 15, 669-78 (2016)). Although these tissues were immediately perfusable and able to survive direct anastomosis in vivo, the scaffold material for the vessel wall remained thick, which limited the interactions between the vasculature and parenchymal cells.

Recent efforts to incorporate perfusable vasculature into engineered tissues have used simple microchannel geometry but lack the requisite vascular density and hierarchical complexity seen in native tissue. Generation of small diameter microvascular networks using current 3D printing processes is restricted by long printing times, lack of perfusion during the printing process, and a lack of applicable biocompatible materials. There is therefore an unmet need for engineered tissues with high density vasculature.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 is a schematic of an exemplary multilayer microvessel fabrication device according to one embodiment.

FIG. 2 is an image of a whole grid of 4-layer microvessel constructs produced according to an embodiment after 7 days of culture and stained for VE-cadherin (green).

FIGS. 3A-3J are maximum intensity projections (MIP) of an embodiment of a confocal z-stack of 8-layer microvessel construct after 7 days of culture stained for VE-cadherin (green) and F-actin (red) of (FIG. 3A) the whole stack, (FIG. 3B) orthogonal view of layers 1-8 as numbered and (FIG. 3C-3J) individual layers.

FIGS. 4A-4D are scanning electron microscopy (SEM) images of (FIG. 4A) the bottom layers numbered 1-6 of an exemplary 8-layer microvessel construct of an embodiment (scale bar 500 μm), (FIG. 4B)-(FIG. 4C) different regions of multilayer microvessel construct with angiogenic sprouts 402 and 404 in layers numbered 1-3 (scale bar 100 μm), and (FIG. 4D) region with holes 406 and 408 connecting layers labeled 1 and 2 in various embodiments (scale bar 100 μm).

FIG. 5 is a histological section of layers labeled 1-3 of an embodiment of an exemplary multilayer microvessel construct with immunohistochemical stain for Ulex europaeus agglutinin (UEA) to label human endothelium (scale bar 200 μm).

FIG. 6 is a SEM image of collagen interface (white arrow) between layers in an embodiment of a multilayer microvessel construct (scale bar 50 μm).

FIGS. 7A-7C are images of a histological section of an embodiment of a multilayer microvessel construct with florescent stain for collagen IV (green) and VE-cadherin (magenta) and numbered layers 1-3 in FIG. 7A (scale bar 200 μm).

FIGS. 8A-8E are maximum intensity projections (MIPs) of an embodiment of a confocal z-stack of 4-layer microvessel construct including human umbilical vein endothelial cells (HUVEC) after 7 days of culture stained for VE-cadherin (green) and F-actin (red) of (FIG. 8A) the whole stack (FIGS. 8B-8E) individual layers (scale bar 500 μm).

FIGS. 9A-9D are MIPs of an embodiment of confocal z-stacks of (FIG. 9A) EGM-1 layer, (FIG. 9B) VBSP 1-layer, and after 7 days of culture (FIG. 9C) whole stack and (FIG. 9D) individual layers for a 4-layer stack with each image (scale bar 500 μm).

FIGS. 10A-10F are MIPs of an embodiment of a z-stack of 4-layer hESC-EC microvessel construct after (FIG. 10A) 7 days of culture, (FIG. 10 B)-(FIG. 10F) 12 days of culture with the full stack shown at (FIG. 10B) and individual stacks at (FIG. 10C)-(FIG. 10F) (scale bar 500 μm).

FIGS. 11A-11D are quantifications of (FIG. 11A) average cross-sectional area per lumen, (FIG. 11B) total luminal volume per field of view, (FIG. 11C) average lumen width and (FIG. 11D) average lumen height in EGM and VBSP in embodiments of single and multilayer microvessel constructs. Mean±SEM. *p<0.05, **p<0.01, ***p<0.001, and ****p<0.0001 determined using two-tailed t-test.

FIGS. 12A-12B are quantifications of average bead velocity at the center of the channel between the fourth and fifth bifurcations for embodiments of an EGM 1-layer, VBSP 1-layer, EGM 4-layer, and VBSP 4-layer vessels. Mean±SEM. *p<0.05, **p<0.01, ***p<0.001, and ****p<0.0001 determined using two-tailed t-test, ns=not significant.

FIGS. 13A-13B are optical microangiography (OMAG) images showing perfusable area of an embodiment of a multilayer microvessel construct with 4 layers showing (FIG. 13A) a projection image with a color-coded scale and depth and (FIG. 13B) a 3D view (scale bar 2 mm).

FIG. 14 is a quantification of mean projected velocity at the inlet of all 4 layers; Mean±SEM. *p<0.05, **p<0.01, ***p<0.001, and ****p<0.0001 determined using two-tailed t-test, ns=not significant.

FIGS. 15A-15E are OMAG-velocimetry (OMAG-V) images of an embodiment of multilayer microvessel constructs with 4 layers showing velocity of each layer near the inlet (scale bar 500 μm) and velocity shown at FIG. 15E.

FIGS. 16A-16B are quantifications of burst pressure testing on (FIG. 16A) acellular multilayer vessels and (FIG. 16B) cellular multilayer vessels where Q1=50 μL/min, Q2=100 μL/min, Q3=200 μL/min, Q4=400 μL/min, and Q5=800 μL/min.

FIG. 17 is a bar chart with the number of upregulated and downregulated differentially expressed genes for all pairwise comparisons from RNAseq analysis of an embodiment.

FIG. 18 is a Venn diagram with the number of differentially expressed genes in an embodiment.

FIG. 19 is a principal component analysis (PCA) plot comparing transcriptional profiles of hSEC-ECs in multilayer microvessel constructs (4 layer μV), single layer microvessel constructs (1-layer μV0, and non-perfusable self-assembled constructs (SA).

FIGS. 20A-20D are heatmaps of the top 100 differentially expressed genes between embodiments of multilayer microvessel constructs and self-assembled constructs normalized to minimum (blue) and maximum (red) of log 2 CPM values.

FIG. 21 is a heat map of all genes that were differentially expressed between any two conditions in an embodiment.

FIG. 22 is a gene ontology analysis for terms upregulated in an embodiment of multilayer microvessel constructs when compared to self-assembled constructs using a 4-layer μV, multilayer microvessel construct, 1-layer μV, single layer microvessel construct, and a self-assembled construct (SA), CPM, counts per million.

FIGS. 23A-23C are MIPs of an embodiment of cleared 4-layer μV graft stained with fluorescein-labeled Griffonia simplicifolia lectin (GSL) of (FIG. 23A) the whole patch (scale bar 1 mm), (FIG. 23B) a partial grid pattern (scale bar 300 μm), and (FIG. 23C) a highly vascularized region (scale bar 200 μm).

FIGS. 24A-24B are depth colored imaging of GSL staining in (FIG. 23A) cleared SA and (FIG. 23B) μV grafts of an embodiment (scale bar 1 mm).

FIGS. 25A-25D are quantifications of (FIG. 25A) GSL+, UEA+ and mT+ lumen density in 4-layer μV and SA grafts based on histological analysis, (FIG. 25B) vessel area density as a percentage of the whole image area, (FIG. 25C) mean velocity within the perfusable vasculature, (FIG. 25D) perfusion relative to the average of the healthy regions in a 4-layer μV, multilayer microvessel construct and SA, self-assembled construct. Mean±SEM. *p<0.05, **p<0.01, ***p<0.001, and ****p<0.0001 determined using two-tailed t-test.

FIG. 26 is a series of optical microangiography (OMAG) and OMAG-based velocimetry (OMAG-V) images of an embodiment providing an analysis of perfusable vasculature in healthy region (left), 4-layer μV graft (middle), and SA graft (right).

FIGS. 27A-27B are images of tissue culture of C2C12s within embodiments of μVs (FIG. 27A) and SA vessels (FIG. 27B) stained for DAPI (blue), F-actin (green), α-actinin (red), and CD31 (far red).

FIGS. 28A-28F are images of LIVE/DEAD staining of embodiments of (FIG. 28A)-(FIG. 28B) μVs and (FIG. 28C)-(FIG. 28F) SA vessels where green is live and red is dead.

FIG. 29 is quantification of cell viability from LIVE/DEAD staining in an embodiment; Mean±SEM. ****p<0.0001 determined using two-tailed t-test.

FIG. 30 is a flow cytometry assessment of hESC-ECs at day 12 of differentiation with a forward and side scatter used to gate main population in an embodiment.

FIG. 31 is flow cytometry assessment of hESC-ECs at day 12 of differentiation of CD31 expression of hESC-ECs on right with overlay of isotype controls in an embodiment.

FIG. 32 is a self-assembled vessel at day 5 stained for DAPI (blue), F-actin (green), and VE-cadherin (far red) in an embodiment.

FIGS. 33A-33B are images of (FIG. 33A) excised heart with patch located on bottom half; graft outlined in blue and (FIG. 33B) excised graft before (left) and after (right) tissue clearing in an embodiment.

FIGS. 34A-34B are images of picrosirius red/fast green stain to (FIG. 34A) 4-layer μV and (FIG. 34B) SA grafts with collagenous hemtoxylin regions, such as the infarct and grafts, stained red/purple and healthy tissue regions stain green.

FIGS. 35A-35D are images of hematoxylin and eosin histological staining of (FIG. 35A) 4-layer μV (scale bar 500 μm) (FIG. 35B) a zoomed in section of a 4-layer μV (scale bar 100 μm), (FIG. 35C) SA grafts, and (FIG. 35D) a zoomed in SA graft in an embodiment.

FIG. 36 is a quantification of infarct size as a percentage of left ventricle area. 4-layer μV, multilayer microvessel construct. SA, self-assembled construct; Mean±SEM. *p<0.05, **p<0.01, ***p<0.001, and ****p<0.0001 determined using two-tailed t-test.

FIG. 37 is an MIP image of cleared SA graft stained with fluorescein labeled Griffonia simplicifolia Lectin (GSL) of an embodiment.

FIGS. 38A-38D are histological analyses of embodiments of 4-layer μV grafts stained for GSL, UEA and mTomato (scale bar 100 μm).

FIGS. 39A-39D are histological analyses of embodiments of SA grafts stained for GSL, UEA and mTomato.

FIGS. 40A-40C are quantifications of GSL+, UEA+ and mT+ lumen size in embodiments of (FIG. 40A) 4-layer μV, (FIG. 40B) SA grafts and (FIG. 40C) histological analysis in healthy and infarcted regions.

FIGS. 41A-41B are depth coloring of the 100 μm of embodiments of the (FIG. 41A) SA and (FIG. 41B) 4-layer μV grafts closest to the host heart, where “bottom” indicates the surface of the graft closest to the host heart and “top” indicates the surface of the graft farthest from the heart.

FIG. 42 is a quantification of perfusion relative to the average of the healthy regions. 4-layer μV, multilayer microvessel construct and SA, self-assembled construct of an embodiment; Mean±SEM. *p<0.05, ****p<0.0001 determined using two-tailed t-test.

### DETAILED DESCRIPTION

Various implementations described herein relate to techniques, systems, apparatuses, and methods for forming thick, perfusable, and highly vascularized tissues. Generally, various implementations are described for modular assembly of individually constructed pieces to create the thick, highly vascularized tissues. In some aspects, these thick, highly vascularized tissues may remodel in vitro and may anastomose to the host vasculature after implantation in vivo.

A modular approach, assembling tissues from smaller components during fabrication, allows precision in creating small-scale features, such as small diameter vessels including capillaries. The flexibility of the modular approach also allows for the re-creation of tissue heterogeneity as individual components may have a different cellular or matrix composition than other components as well as different patterning. Further, the creation of smaller components allows for a decrease in the nutrient-deficient periods within the bulk matrix in tissue fabrication as each layer can be rapidly fabricated. In some aspects, each layer is fabricated in parallel. That is, multiple layers may be fabricated at the same time using different devices or other platforms. Automation may be used for the alignment and fabrication of each layer. For exemplary purposes the process will be described using soft lithography patterning, though one of ordinary skill in the art would understand that other fabrication methods may also be used.

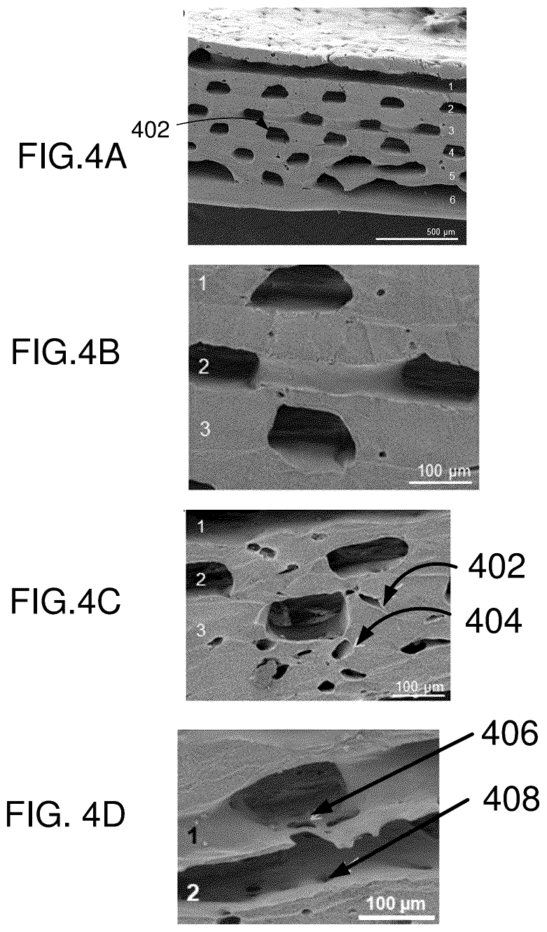

In some aspects, these modular tissues may be formed by stacking multiple layers including 1-100, 1-75, 1-50, 3-40, 3-35, 4-20, 5-15, or any subset thereof of patterned vasculature within a re-modellable collagen hydrogel to form a perfusable integrated construct. Each layer of the patterned vasculature may be the same or different. In some aspects, a single layer may provide some or all of a layer of vasculature, allowing combined layers to form more complex structures, or portions of connecting structures. For example, in one aspect a first layer may include a first portion of a vasculature structure, with a second layer including a second portion of a vasculature structure such that when the first layer and the second layer are stacked, the first portion of a vasculature structure in a first layer and a second portion of a vasculature structure in the second layer form at least a part of a combined structure. In some aspects, a combined structure may be formed through the combination of two or more layers. For example, in some aspects, the bottom surface of one layer and the top surface of the immediately lower layer, where the bottom surface of the one layer and the top surface of the immediately lower layer are in face sharing contact, may be mirror images. In other aspects the bottom surface of one layer may contain an opening or lumen that connects with an opening or lumen in a top surface of the next layer. In other aspects the bottom of one layer and the top of the immediately lower layer may be complementary such that the bottom or top has a positive projection, that is, a portion that is raised or otherwise protrudes away from the body of the layer, and the corresponding bottom or top has a negative or recessed portion that fits with the positive projection of the opposite layer. For example, the top surface of the immediately lower layer may have a raised portion and the bottom surface of an upper layer may have a recessed portion that fits with the raised portion or vice versa. These and other features may be used together or separately depending on the layer or purpose of the engineered tissue. While two layers are referenced, such patterns may continue through the plurality of layers used to form the modular tissues. In some aspects, a plurality of layers such as three, four, five, six, seven, eight, nine, ten, at least twenty, at least thirty, at least forty, at least fifty or more layers may together form a structure where the combination of different layers produces a perfusable tertiary structure. In some aspects, the use of a modular systems may allow the generation of integrated, perfusable, highly vascularized tissues with thicknesses of 400 μm or greater, for example 10 μm to 1 cm or greater, 10 μm to 3 mm, 500 μm to 2 mm, 10 μm to 1 mm, 10 μm to 500 μm, 20 μm to 100 μm, 10 μm, 500 μm, 2 mm, 2 mm or greater, or any number in between. In some aspects, the thick, highly vascularized tissues can remodel in vitro and anastomose to the host vasculature after implantation in vivo. For example, as shown in FIG. 4A, the bottom of a first layer 1 is in face sharing contact with the top of the second layer 2. The bottom of the second layer 2 is in face sharing contact with the top of the third layer 3. As shown at 402, a first portion of a lumen is formed in layer 4 and a second portion of the lumen is formed in layer 5.

In various examples, the modular approach described herein allows for the fabrication of large-scale, perfusable tissues. Each layer of a microvessel construct (microvessel) may be fabricated through a variety of mechanisms including soft lithography patterning, modular molding, and 3D or other bioprinting.

Each layer may be formed using one or more different materials. For example, in some aspects, one or more layers may be formed using collagen and collagen hydrogels. The type of collagen used may depend on the particular tissue being built as different tissue types may be better suited to different protein amounts or different types of collagen sources. Further, different tissue layers may use different types of collagen. For example, a first layer may be made of a first collagen and a second layer may be made of a second collagen and so forth where each layer may be the same or different. For example, larger scale structures may require higher concentrations of collagen relative to smaller structures to provide additional mechanical support and compensate for additional pressures due to the size of the structure. For some cells, such as cardiomyocytes, a lower concentration of collagen may allow for an increase in cell-cell communication. The collagen may be used alone or in combination with other materials such as endothelial cells, stem cell derived endothelial cells, myoblasts, cardiomyocytes, adipose cells, myocytes, smooth muscle cells, skeletal muscle cells, stromal cells, and other appropriate cells depending on the type of tissue being engineered or the type of tissue being replaced. Exemplary cell types include human umbilical vein endothelial cells (HUVECs), human stem cell derived endothelial cells (hESC-ECs), C2C12 mouse myoblasts, and the like. The modular system permits the fabrication of many layers in parallel which can then be assembled, increasing tissue survival in comparison to current techniques. Further, the modular system permits heterogeneity within the highly vascularized tissues, allowing for different compositions to be used in different layers.

The layers may be formed using a variety of techniques including soft lithography, modular molding, anastomosis-based remodeling, needle subtraction, subtractive sacrificial scaffolds, and 3D or other bioprinting. In soft lithography, any type of elastomeric compound may be used to form the stamp, for example, polydimethylsiloxane (PDMS), a tri-allyl-tri-azine:tri-thioltriacine 4:3 mixture, tri-allyl-tri-azine:tetra-thiolpentaerythritol 2:3 mixture, as well as other polymeric materials such as photocurable perfluoropolyethers, or cyclicolefin copolymer.

In one aspect, the tissue layers may be constructed using soft lithography and injection molding as described in Example II, below. Various implementations of the present disclosure are directed to improvements in the technical field of tissue engineering. The modular approach described herein allows for the generation of large, three-dimensional tissues with perfusable networks of vasculature. In some aspects, the perfusable vasculature networks exhibit remodeling while maintaining a patterned, open-lumen architecture. Further, the engineered tissues increase gene expression indicative of vascular development and angiogenesis in comparison to other types of engineered tissue.

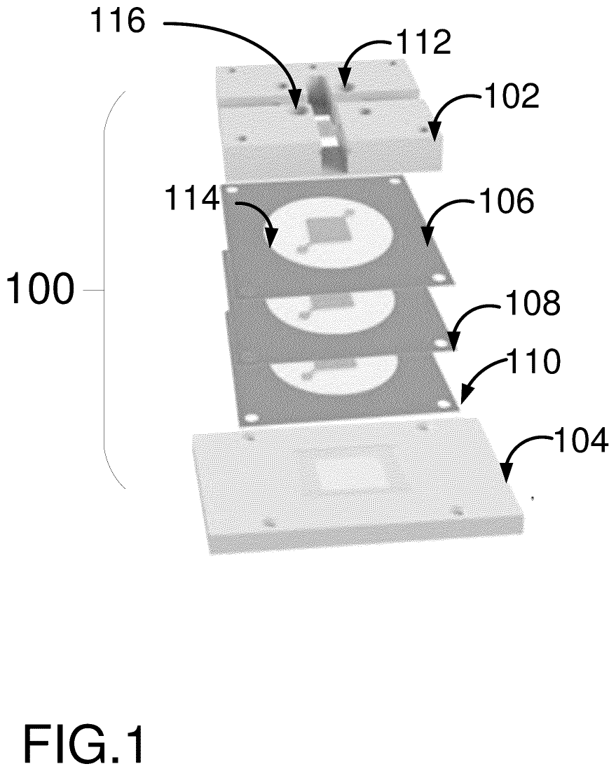

FIG. 1 is a schematic of an exemplary multilayer microvessel fabrication device 100. The fabrication device 100 includes a top housing 102, a bottom housing 104, and a plurality of patterned membranes 106-110. A designated inlet 112 and outlet 116 allows for perfusion through the constructs and additional removable pieces allow for media access on the surface of the construct after fabrication. While three patterned collagen membranes are shown, more or fewer collagen membranes may be used. The patterned membranes may be made of collagen. In other aspects, the patterned membranes may be constructed without collagen membranes but with cells seeded into the lumen. In some aspects, cells may be combined with collagen prior to fabrication. Each patterned membrane may be made of the same or different materials.

Microvessel fabrication devices such as microvessel fabrication device 100 may be constructed by any method generally used. In some aspects, an acrylic device with holes for inlets and outlets may be fabricated. In some aspects, one or more of the top and/or bottom housing may be removable. In other aspects one of the top or bottom housing may be fixed. The device may be uncoated or coated with compositions to provide a surface for collagen binding such as positively charged extracellular matrix proteins or polymers. Such compositions include, polyethylenimine, glutaraldehyde, and polylysine, alone or in combination.

To form top and bottom collagen pieces, collagen hydrogel may be injected and molded between a flat elastomeric stamp and the respective top or bottom housing device. Collagen membranes such as collagen membranes 106-110 may be fabricated by gelling collagen within a form between one flat piece and one patterned piece of an elastomeric stamp, between two flat pieces, or between two patterned pieces of an elastomeric stamp. The same or different elastomeric stamps may be used with each layer. Generally, any type of elastomeric compound may be used to form the stamp, for example, polydimethylsiloxane (PDMS), a tri-allyl-tri-azine:tri-thioltriacine 4:3 mixture, tri-allyl-tri-azine:tetra-thiolpentaerythritol 2:3 mixture, as well as other polymeric materials such as photocurable perfluoropolyethers, or cyclicolefin copolymer. In some aspects, the membranes may be anchored in a frame such as a chemically treated plastic sheet.

While any pattern may be used in the patterned stamp, in some aspects the stamp may include a pattern reflecting the desired vascular diameter for some or all of the engineered tissue. In some embodiments, the desired vascular diameter is 100 μm or less. In some aspects it is between capillary size (˜10 um) to meta arterioles (˜500 um) or any fraction thereof. The vascular diameter may be 10 μm, 20 μm, 30 μm, 40 μm, 50 μm, 60 μm, 70 μm, 80 μm, 90 μm, 100 μm, 150 μm, 200 μm, 250 μm, 300 μm, 350 μm, 400 μm, 450 μm, or 500 μm.

The patterned stamp may be used to form lumens throughout the engineered tissue. In some aspects, separation of lumens in the z-direction may be altered by using thinner or thicker membranes. Lumen separation in the xy-plane, vessel diameter, and planar morphology of the vasculature may be modified by using a different geometry in the lithography process. Further, additional modification towards vascular complexity, such as patterning of capillary-sized vessels, could be added through other methods, for example, collagen ablation.

The layers of the multilayer microvessel constructs may be made with collagen alone or in combination with one or more different cell types such as endothelial cells, stem cell derived endothelial cells, myoblasts, and stromal cells. Exemplary cells include human umbilical vein endothelial cells (HUVECs), human stem cell derived endothelial cells (hESC-ECs), C2C12 mouse myoblasts, and the like. In some aspects, the collagen is mixed with cells prior to fabrication. In some aspects collagen membranes are assembled and seeded with one or more cell types at the desired densities. Such seeding may occur at various stages of the process. In some examples, the seeding may occur during collagen membrane formation. In other aspects, seeding may occur after collagen membrane formation. In additional aspects, the seeding may occur before, during, and/or after culturing the multilayer microvessel constructs with cell media. The seeding may be accomplished with one or more concentrations of one or more types of cells. For example, the one or more types of cells to be seeded may be seeded in concentrations between 0 to 50 million/mL including 2.5 million/mL to 30 million/mL, 3 million/mL to 10 million/mL, 2.5 million/mL, 30 million/mL or any subset thereof. Such concentrations may apply to the total amount of cells, or the amount of each cell type. Each layer may independently use the same or different types of cells in the same or different concentrations, allowing for heterogeneity within the matrix.

While the height of the fabrication device with the plurality of collagen membranes may have a variable height depending at least in part on the number of collagen membranes being used, in an exemplary embodiment to manufacture a multilayer microvessel construct with four layers, three collagen membranes sandwiched between the top and bottom housing devices resulted in a collagen construct with a height of 1.4 mm-1.5 mm. In another example, an 8-layer microvessel construct was formed using 7 collagen membranes between the top and bottom housing devices has a resulting height of 2.2 mm to 2.3 mm. In some aspects, each layer may be between 1 mm and 5 mm, in some aspects they may be 3 mm.

Once the membrane is formed, a punch or other instrument may be used to create a hole for the inlet and outlet. While the holes may be included on any portion of the collagen membranes and the holes in one more collagen membranes may be the same or different, in some aspects the holes may be created along the long axis of the oval membrane shape 114 shown in FIG. 1. In some aspects, the holes may be at opposite ends of a collagen membrane to form an inlet and an outlet. A designated inlet and outlet may allow for perfusion through the constructs and additional removable pieces may allow for media access to the surface of the construct after fabrication. In some aspects, each inlet in the stack of collagen membranes may align along a same axis, such as an axis parallel to gravity and/or perpendicular to the ground during fabrication. In some aspects, each outlet in the stack of collage membranes may align along the axis. In other aspects, some or none of the inlets and/or outlets may align along the axis.

By fabricating each thin layer separately, there is flexibility to pattern different vessel geometries, modify the thickness of the membranes, and incorporate heterogeneous vascular and parenchyma cell types to support composite tissue function. Further, the modular system described herein allows for the creation of multiple layers simultaneously that may be assembled into a larger tissue. As each tissue may be fabricated using only a small amount of time, the likelihood of tissue survival is increased.

After fabrication and before or after cell seeding, the microvessel constructs are cultured with appropriate cell media as described in further detail in Example II, below. For example, endothelial cells may be cultured with one or more of endothelial cell growth medium, vascular endothelial growth factor (VEGF), basic fibroblast growth factor (bFGF), phorbol 12-mysterate 13 acetate (PMA), proangiogenic factors (VBSP), vascular basal medium, and sphingosine-1-phosphate. Appropriate growth mediums may be used with other cell types. During fabrication, the collagen layers integrate into a cohesive construct. In some aspects, the multilayer microvessel constructs prepared as described herein may remodel the surrounding collagen matrix to undergo angiogenic sprouting into the bulk collagen while maintaining high architectural fidelity.

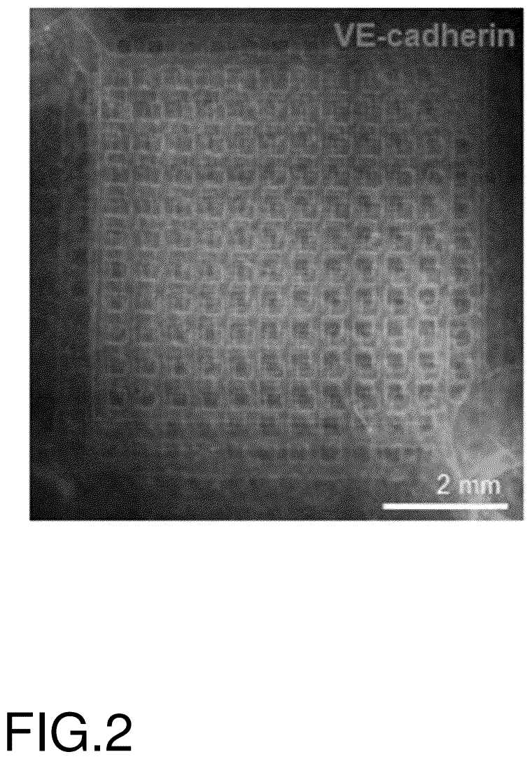

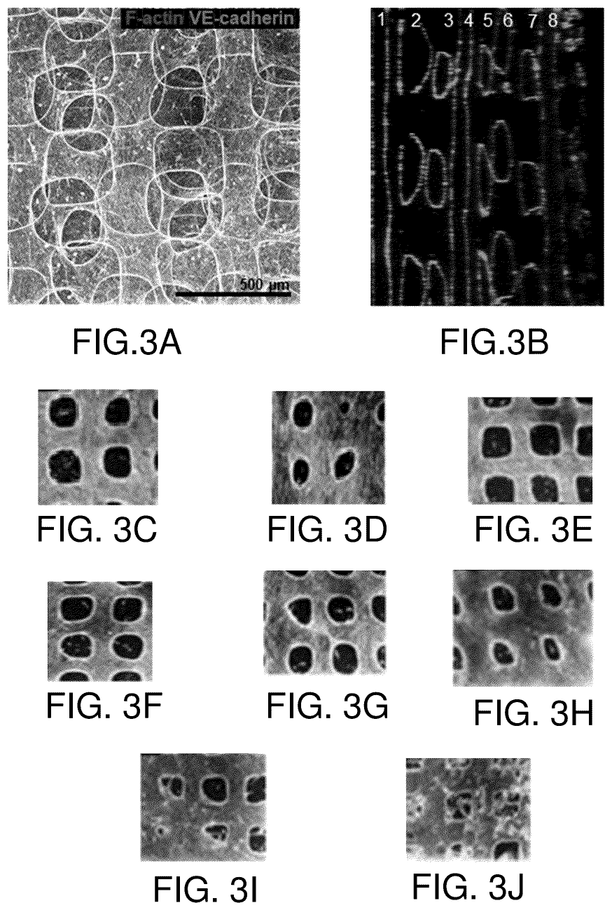

As shown by the staining for VE-cadherin in FIG. 2, after 7 days of culture, a multilayer microvessel construct including 4 layers maintained its initial network geometry with patent endothelial-lined lumens in each of the four layers. Eight-layer microvessel constructs were successfully seeded through the same inlet and outlet and maintained patent endothelial-lined lumen layers throughout 7 days of culture as shown in FIGS. 3A-C. The 8-layer stack was stained for VE-cadherin (green) and F. actin (red). FIG. 3A is a maximum intensity projection of the whole stack, an orthogonal view is provided at 3B and of individual layers in FIGS. 3C-3J (scale bar, 500 μm). The scanning electron microscopy images shown in FIGS. 4A-4C reveal that 8-layer microvessel constructs have a robust structural architecture of open lumens and high vascularity. The vascularized portion of the tissue has 20 patterned lumens per mm², covering over 25% of the cross-sectional area. The maximal spacing between adjacent lumens was less than 380 μm in the xy-plane, which was mainly determined by the selected geometry of the patterned network. As shown in FIG. 4A, adjacent lumens in the z-direction were separated by less than 100 μm. Zoomed view as shown in FIGS. 4B-C show the formation of a smooth and robust endothelium lining the vasculature including angiogenic sprouts 402 and 404 (scale bar, 100 μm).

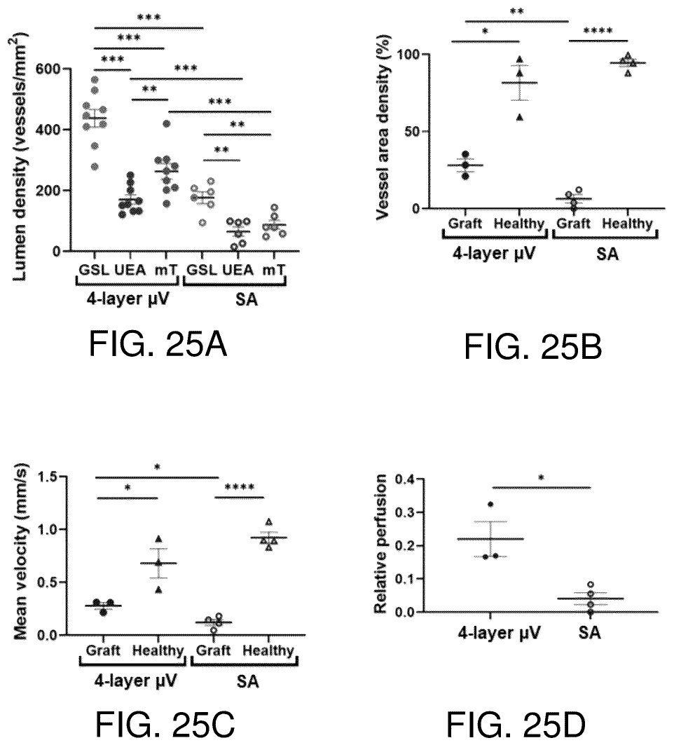

In some aspects, the multilayer microvessel constructs as described herein may remodel their surrounding collagen matrix to undergo angiogenic sprouting into the bulk collagen while maintaining high architectural fidelity. In regions with high vascular remodeling, sprouts with open lumens branched off the patterned network into the surrounding collagen as shown in FIG. 25A.

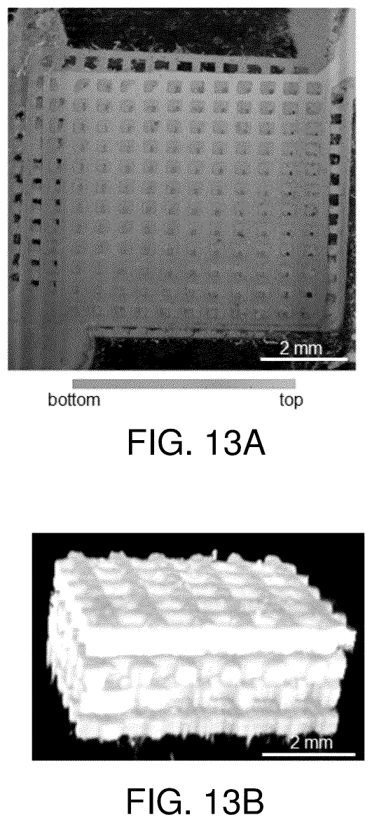

Earlier attempts to create engineered tissue resulted in low tissue prefusion, leading to tissues with a thickness of <500 μm and poor integration with host vasculature. As shown in Example V, below, various engineered tissues described herein had increased total vessel area in the multilayer constructs in comparison to each individual layer. Perfusion of intralipid through the constructs demonstrated that each layer was perfused in its entirety and perfusion dynamics near the inlet showed similar perfusion throughout each layer suggesting evenly distributed perfusion (FIGS. 13A-15E).

Implementations of the collagen layers of the engineered tissue as described herein may integrate into a cohesive construct. Such cohesiveness was confirmed via burst pressure testing as described in further detail Example VII, below. The pressure drop was compared in both cellular and acellular 4 layer microvessel constructs perfused with media at a range of flow rates. With flow rates above 50 μl/min, endothelized multilayer vessels exhibited a step-like increase in pressure following each increment in flow. Thus, the presence of cells may contribute to the sealing and stability of the multilayer structure.

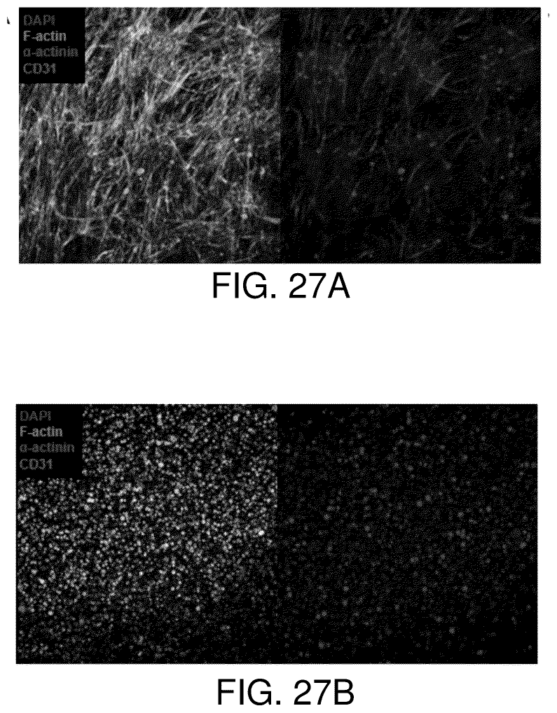

The multilayer microvessel tissues may additionally support the survival of metabolically active tissue cells. For example, as described in Example VIII, below, after seven days, C2C12 mouse myoblasts in a 4-layer microvessel tissue had a more robust cellular population and a 3-fold increase in viability in comparison to self-assembled microvessel tissues as shown in the live/dead staining in FIGS. 27A-29.

The multilayer microvessel tissues may upregulate positive regulation of angiogenesis, positive regulation of vasculature development, cell-matrix adhesion, second-messenger-mediated signaling, positive regulation of cell migration, and other vascular remodeling and stability in comparison to self-assembled constructs (FIG. 22).

Engineered tissue holds promise for replacement, repair, or amelioration of biological tissues and shows promise in generating biomimetic tissue for disease modeling, drug development and regenerative medicine. Tissues may be developed using autologous or allogenic cells for particular purposes such as reconstruction, muscle and other tissue replacement, and organ replacement. These tissues may also be useful in disease modeling, allowing for the creation of genetic, phenotypic, and physiological, tissues created for particular protocols or tests for a specific patient. The use of highly vascularized tissues provides an improved model for specific processes or diseases of human biology. Such models may additionally assist in diagnosis and the development of new and improved therapies.

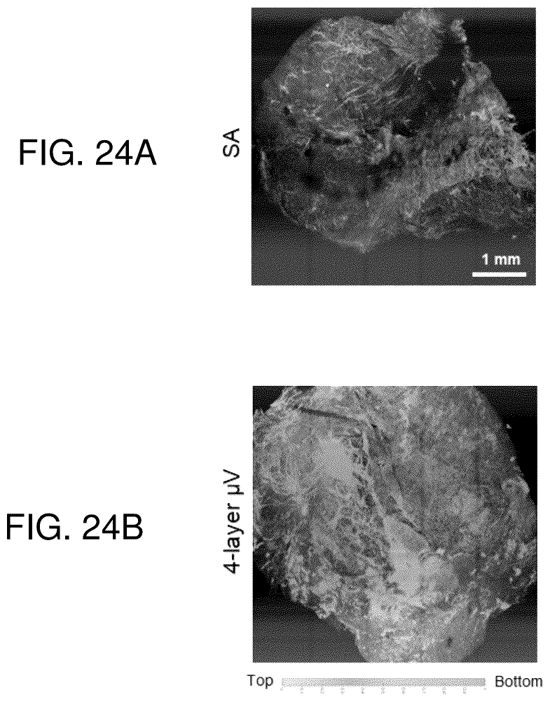

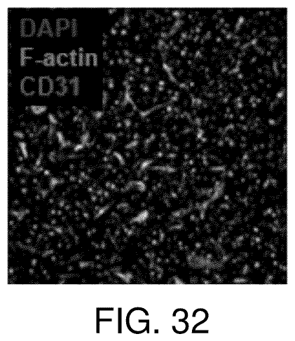

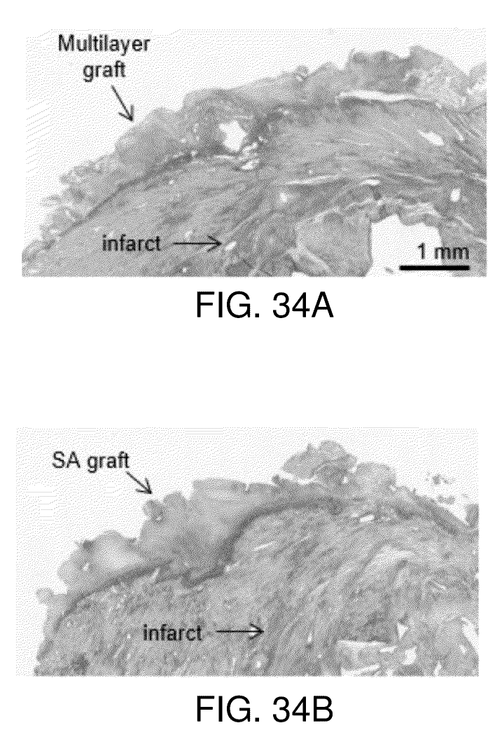

The multilayer microvessel tissues described herein may be used as grafts for damaged tissues or portions of tissues. The multilayer microvessel tissue with the desired tertiary structure and perfusability may be sutured over the damaged area. In some aspects, the multilayer microvessel tissue and the host tissue may integrate in the area between and/or surrounding the multilayer construct and vice versa such that vasculature from the host tissue undergoes angiogenesis into the multilayer tissue construct and vice versa. For example, in acute myocardial infarction or chronic cardiac diseases, significant cardiomyocyte death may occur with minimal regenerative capacity, leading to scar formation with deficient vasculature and declined heart function, contributing to eventual heart failure. As an example, highly vascularized thick tissues created as described above were used to repair heart muscle in rats. As shown in FIG. 32, SA constructs formed capillary tubes, but most tubes remained either isolated or connected in very small regions with no perfusion. In the tissue sample including the multilayer graft shown in FIG. 34A, there are high levels of cellularity, indicating tissue remodeling. In contrast, as shown in FIG. 34B, the SA constructs appear to have more sparse and singular cellular organization. Further the microvessel grafts had a greater depth and density of vascularization in comparison to the self-assembled grafts as shown in FIGS. 24A-B and 41B. Additionally, multilayer microvessel grafts have significantly greater density of both GSL-positive and UEA-positive lumens when compared to self-assembled grafts, indicating higher vascular integration of multilayer microvessel constructs with the host at early timepoints. Further, the multilayer microvessel grafts have higher lumen density, vessel area density, mean velocity and relative perfusion in comparison to SA grafts.

The modular microvessel tissues described herein allow for the creation of small-scale features and heterogeneity in thick engineered tissues and provides extensive vascularization, allowing for increased complexity and scale. By using a modular approach, it is possible to increase the diversity of shape, size, and cell type in the structure. The use of such an approach forms cohesive tissues in which the layers integrate into a whole, allowing for perfusion, through and around the tissue.

In order that the invention described herein may be more fully understood, the following examples are set forth. Those of skill in the art should, in light of the present disclosure, appreciate that many changes can be made in the specific embodiments which are disclosed and still obtain a like or similar result without departing from the spirit and scope of the invention. Further, it should be understood that these examples are for illustrative purposes only and are not to be construed as limiting this invention in any manner.

### EXPERIMENTAL EXAMPLES

### Example I: Cell Culture and hESC-EC Differentiation

Human umbilical vein endothelial cells (HUVECs), human embryonic stem cell derived endothelial cells (hESC-ECs), and C2C12 mouse myoblasts (ATCC) were used in this study. HUVECs were cultured in EGM-2 and used at passage 5-6. C2C12s were cultured in DMEM supplemented with 10% FBS. hESC-ECs were differentiated from a genetically modified mTmG-2a-Puro dual-reporter line of RUES2 (Rockefeller University, NIH 0013) hESCs29. The hESCs were only used in their untreated state in which the mTmG-2a-Puro transgene causes the cells to stably express tdTomato red fluorescent protein (mT). Undifferentiated hESCs were maintained on Matrigel (BD Biosciences) in mTeSR Plus (Stemcell Technologies). To differentiate the hESCs into hESC-ECs, cells were treated with high Activin A and low BMP4 (Redd, M. A. et al. Nat Commun 10, 584 (2019); Rayner, S. G. et al. Adv Healthc Mater 7, e1801120 (2018)). Briefly, hESCs were replated into Matrigel coated 24-well plates at a density of 200K/well in mTeSR Plus with 10 μM Rock inhibitor (Stemcell Technologies) and 1 μM CHIR-99021 (Cayman Chemical). After 24 hours, hESCs were induced in RPMI (Gibco) with 100 ng/mL Activin A (R&D), 1× Matrigel and 1×B-27 Supplement, minus insulin (Thermo Fisher). The time of induction is referred to as day 0. 18 hours after induction, the media was changed to RPMI with 5 ng/mL BMP4 (R&D), 1 μM CHIR-99021, and 1×B-27 Supplement, minus insulin. On day 2 at 42 hours after induction, the media was changed to StemPro-34 SFM (Invitrogen) with 100 ng/mL VEGF (Peprotech), 5 ng/mL bFGF (Peprotech), 10 ng/mL BMP4, 50 μM Ascorbic Acid (Sigma), 2 mM L-Glutamine (Invitrogen), and 400 nM monothioglycerol (Sigma). The media was unchanged for 72 hours. On day 5, the media was changed to StemPro with 10 μM Rock inhibitor and 2 mM L-Glutamine. One hour after the media change, cells were harvested using versene (Thermo Fisher) with 0.25% trypsin (Thermo Fisher). Cells were replated into 0.2% gelatin coated 10 cm dishes at a density of approximately 104/cm2 in EGM-2 with 20 ng/mL VEGF, 20 ng/mL bFGF and 1 μM CHIR-99021. Media was changed to EGM-2 with 20 ng/mL VEGF and 20 ng/mL bFGF at day 7 then changed every other day until the cells were harvested for use via trypsinization. hESC-ECs were used on day 10-12 of differentiation for all experiments. To assess purity, an aliquot of hESC-ECs was fixed in 4% PF then stained with 1:5 mouse anti-human CD31 FITC (BD Biosciences, 560984) in PBS for 45 minutes. Cells were assessed by flow cytometry on a Canto II system and FlowJo Software.

### Example II: Fabrication and Culture of Perfusable Multilayer Microvessel Constructs

Multilayer microvessel constructs were generated from modular fabrication and assembly of single layer microvessel constructs. Each single layer was formed in collagen through soft lithography and injection molding by following Zheng, Y. et al. Proceedings of the National Academy of Sciences of the United States of America 109, 9342-7 (2012).

First, custom made acrylic housing devices that contained holes for inlets and collagen injection were milled and fabricated. The top housing devices were constructed to have removable portions to allow for additional media access upon submersion. Plastic shims (AccuTrex) of thickness ˜200 μm were cut into 50×50 mm squares. Holes were cut in the center and four corners. A circular, 18 mm diameter, center hole was used for initial characterization experiments in FIG. 1, and an oval, 25 mm×18 mm, center hole was used for the remaining experiments, due to greater ease of fabrication. To assist in correct placement of the pattern during fabrication, lines were drawn on the plastic shims to mark the long and short axis of the oval cutout. Then, flat and patterned PDMS pieces were prepared, with patterned pieces having a 13×13 grid network that protruded 110 μm. Inner surfaces of plastic shims and housing devices (excluding the removable pieces of the top housing device) were coated with 1% polyethylenimine (Sigma) and 0.1% glutaraldehyde (Sigma) to provide a surface for collagen bonding. Once all the components were prepared, 6 mg/mL collagen was prepared by mixing 15 mg/mL type I collagen (extracted from rat tails) diluted in 0.1% acetic acid, 1 M NaOH (20 mM final concentration), 10× Medium 199 (ThermoFisher), Matrigel (1:60 dilution), and EGM-2. ECs were added to the liquid collagen to reach either 3 million/mL for HUVECs or 2.5 million/mL for hESC-ECs and the mixture was maintained on ice for fabrication. For C2C12 experiments, C2C12s were also added to the liquid collagen at a density of 30 million/mL.

Top and bottom collagen pieces were fabricated as previously described in Zheng, Y. et al. Proceedings of the National Academy of Sciences of the United States of America 109, 9342-7 (2012)). For the top piece, collagen hydrogel was injected and molded in the top housing device against a PDMS stamp with a positive feature of a 13×13 grid network with a vascular diameter of ˜110 μm. A designated inlet and outlet allow for perfusion through the constructs and additional removable pieces allow for media access on the surface of the construct after fabrication. For the bottom collagen piece, collagen hydrogel was injected and molded between a flat PDMS stamp and the bottom housing device with coverslip at the bottom imaging window. Collagen membranes were fabricated by gelling collagen within the large circular cutout of the plastic shims in between one flat piece and one patterned piece of PDMS. To ensure proper alignment of patterned grids between layers, each corner of the square grid pattern was aligned with the four axis lines previously drawn onto the plastic shims. A 2 mm biopsy punch was used to cut holes in the membranes for the inlet and outlet. These holes were punched along the long axis of the oval membrane shape and in line with channels which extend from opposite corners of the grid pattern.

To fabricate multilayer microvessel constructs with 4 layers, 3 collagen membranes were sandwiched between the top and bottom housing devices as shown in FIG. 1, that is, the top housing device formed a first layer and the first through third collagen membranes formed second, third, and fourth layers, resulting in a collagen construct with four vascularized layers and a bottom flat layer with a height of 1.4 mm-1.5 mm. For 8-layer microvessel constructs, 7 collagen membranes were added, resulting in a height of 2.2-2.3 mm. The four holes cut into the corners of the plastic shims were used as anchor points, using dowels to hold membranes in the correct place while stacking. The stacked multilayer microvessel constructs share the same inlet and outlet ports and have open lumens that are completely perfusable. Single layer microvessel constructs were similarly constructed without collagen membranes. HUVECs or hESC-ECs were seeded into the lumen at 107/mL for 10 μL and allowed to attach for 30 min to 1 hr before moving into the culture dishes. For experiments for comparison, self-assembled constructs were created by gelling EC mixed collagen hydrogel in 12 mm×12 mm×1.5 mm PDMS wells and submerged in media for culture.

After fabrication and cell seeding, all microvessel constructs were placed into deep 10 cm dishes that snugly fit the housing device. EGM-2 was added to submerge the microvessel construct to approximately half the height of the housing device. Microvessel constructs were then cultured without motion overnight to allow for cell attachment. After 24 hours, microvessel constructs were moved to a custom-made plate rocking system that tipped the culture dishes 15° every 3 hours. The dishes were aligned such that the inlets and outlets were perpendicular to the axis of rotation, causing media to accumulate in the lower inlet/outlet then perfuse through the microvessel construct to the other inlet/outlet after tipping. The media was removed from the inlets and outlets and replaced every 24 hours. For hESC-EC microvessel constructs and self-assembled constructs, 20 ng/mL VEGF and 20 ng/mL bFGF were added directly after fabrication. HUVEC microvessel constructs were either cultured without additional supplementation or with 50 ng/mL VEGF, 50 ng/mL bFGF, 50 ng/mL phorbol 12-myristate 13 acetate (PMA) (Sigma), and 300 nM sphingosine-1-phosphate (Tocris) which was added to the media 24 hours after fabrication.

At 7 days of culture, multilayer microvessel constructs maintained their initial network geometry with patent endothelial-lined lumens in each of the 4 layers (FIG. 2). To determine the robustness of potential scale up, 2.2 mm-thick constructs with 8 layers of perfusable vasculature were generated (see FIG. 3 A-J and FIG. 4A). The 8-layer microvessel constructs were successfully seeded through the same inlet and outlet and maintained patent endothelial-lined lumens in all 8 layers throughout 7 days of culture (FIG. 3). Additionally, scanning electron microscopy (SEM) imaging revealed that 8-layer microvessel constructs had a robust structural architecture of open lumens and high vascularity (FIG. 4A). This vascularized portion of the tissue had 20 patterned lumens per mm², covering over 25% of the cross-sectional area. The maximal spacing between adjacent lumens was less than 380 μm in the xy-plane, which was mainly determined by the selected geometry of the patterned network. More importantly, adjacent lumens in the z-direction were separated by less than 100 μm, demonstrating that close vessel proximity can be achieved between stacked layers. Zoomed views also showed the formation of a smooth and robust endothelium lining this vasculature (FIG. 4B).

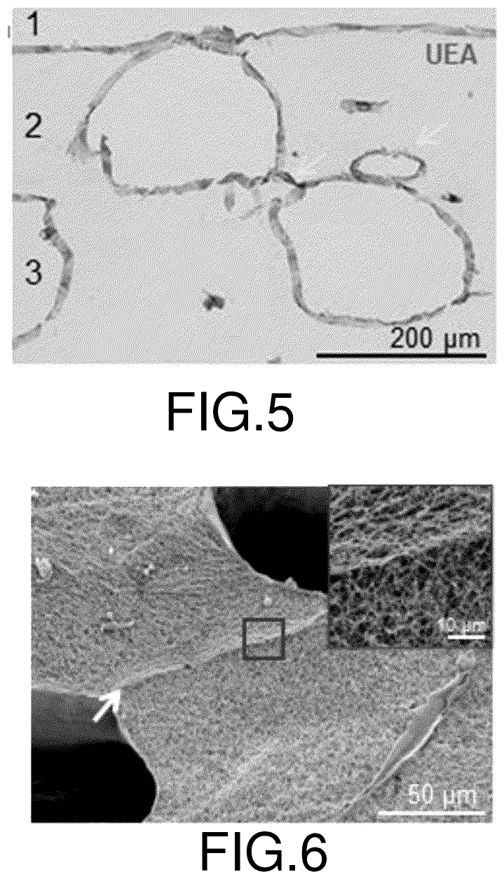

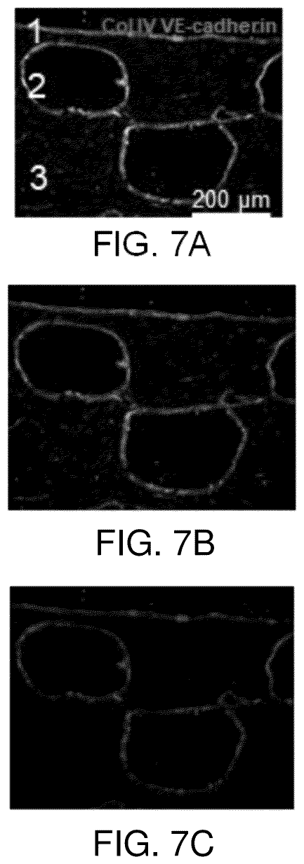

The created multilayer constructs re-modelled the surrounding collagen matrix to undergo angiogenic sprouting into the bulk collagen while maintain high architectural fidelity after 1 week of culture. As shown in FIGS. 4B, 4C, and 5, the amount of vascular remodeling varied from region to region. In regions with high vascular re-modeling, sprouts with open lumens branched off the patterned network into the surrounding collagen as shown in FIGS. 4C and 5. In regions of low vascular remodeling, the network maintained its initial pattern and the endothelium was smooth and adhered in the patterned collagen lumens as shown in FIG. 4B. In some regions, the vascular remodeling formed endothelial lined pores 406 and 408 connecting the two layers as shown in FIG. 4D. Ultrastructural SEM imaging showed that collagen layers formed a clear, gap-free interface with slight compression of the collagen fibers as shown in FIG. 6. Additionally, basement membrane protein collagen IV was expressed on the endothelialized walls of both the patterned lumens and sprouts as shown in FIG. 7A-C. These findings demonstrated that each collagen layer is structurally robust but can also be assembled and integrated into a perfusable cohesive construct.

### Example III: Immunofluorescent and Histological Staining, Imaging and Analysis

All immunofluorescent and histological analysis was performed on multilayer and single layer microvessel constructs that were cultured for 7 days. At the endpoint, 4% PF was perfused through the inlet and added onto the exposed collagen surface and left to fix for 30 minutes. Microvessel constructs were then washed with PBS three times in the same manner. For 3D immunofluorescent imaging and analysis, HUVEC and hESC-EC microvessel constructs were stained for VE-cadherin and F-actin and C2C12 microvessel constructs were stained for F-actin, α-actinin, and CD31. Briefly, microvessel constructs were permeabilized and blocked with 0.5% Triton X-100 (Sigma) and 2% BSA (Sigma) in PBS for 1 hour. Microvessel constructs were then stained with 1:100 Alexa Fluor 647 Phalloidin (Thermo Fisher, A22287), 1:50 mouse anti-human CD144 PE (Thermo Fisher, 12-1449-82), and 1:250 Hoechst for HUVEC microvessel constructs or 1:100 Alexa Fluor 488 Phalloidin (Thermo Fisher, A12379), 1:50 mouse anti-human CD144 APC (Thermo Fisher, 17-1449-42), and 1:250 Hoechst for mT-hESC-EC microvessel constructs. C2C12 microvessel constructs were stained with primary antibodies 1:100 mouse sarcomeric alpha actinin (Thermo Fisher, MA122863) and 1:100 rabbit anti-human CD31 and then secondary antibodies 1:100 Alexa Fluor 488 Phalloidin (Thermo Fisher, A12379), 1:250 Hoechst, 1:100 goat anti-mouse Alexa Fluor 568 (Thermo Fisher, A11004), and 1:100 donkey anti-rabbit Alexa Fluor 647 (Thermo Fisher, A31573). For 8-layer tissues, RapiClear (SunJin Lab, RC149001) was added to the inlet and to the surface of the exposed collagen at least 1 hour prior to imaging to allow for increased visualization through collagen throughout the whole thickness of the constructs. Large images containing the whole grid were taken on a Nikon Eclipse Ti2 widefield microscope. Smaller view 10×3D confocal z-stacks were taken on a Nikon A1R confocal microscope at random locations within the grid pattern.

FIJI image processing software (Fiji: an open-source platform for biological-image analysis Nat. Methods, 9 (2012), pp. 676-682, 10.1038/nmeth.2019) was used to create maximum intensity projection images and for quantification of luminal volume and cross-sectional measures from the 10× confocal z-stacks. For luminal volume analysis, the open luminal area was quantified within each z-plane then multiplied by the thickness between individual slices. The luminal volume represents the luminal volume within one 10× z-stack field of view, not the total construct. For cross sectional analysis, orthogonal views of z-stacks were used to measure height, width, and area of individual lumens. IMARIS Viewer was used to create orthogonal images of 8-layer microvessel constructs. For histological assessment, microvessel constructs were embedded in OCT compound (Fisher Scientific) then sectioned into 10 μm slices using a CryoStat. Sections were stained for either Ulex Europaeus Agglutinin I (UEA) or Col IV and VE-cadherin. For UEA staining, slides were blocked with 5% Normal Goat Serum (Jackson ImmunoResearch) for 1 hour, stained with 1:100 biotinylated UEA (Vector Laboratories, B-1065-2) overnight, stained using a VectaStain Elite ABC-HRP kit (Vector Laboratories, PK-6100) and SIGMAFAST DAB tablets (Sigma, D4293). For Col IV and VE-cadherin staining, slides were permeabilized and blocked with 0.5% Triton X-100 and 2% BSA for 1 hour, stained with 1:100 rabbit anti-collagen IV (Abcam, ab6586) overnight, then 1:100 mouse anti-human CD144 APC (Thermo Fisher, 17-1449-42), 1:100 Alexa Fluor 488 goat anti-rabbit, and 1:250 Hoechst for 1 hour then imaged on a Nikon TiE Inverted Widefield Fluorescence Microscope with Yokogawa W1 spinning disk head.

### Example IV: Scanning Electron Microscopy Preparation and Imaging

Scanning electron microscopy (SEM) was performed on 8-layer microvessel constructs that were fixed on day 7 as described above. Following 4% PF fixation, the entire collagen construct containing the microvessel constructs was removed from the housing device and plastic shims. The portion of the construct that contained the grid pattern was cut out using a razor blade, then cut in half for a cross-sectional slice through the lumens. Next, constructs were fixed in ½ strength Karnovsky's fixative (2.5% glutaraldehyde, 2% PF in 0.1M sodium cacodylate buffer, pH 7.3) overnight at 4° C. Constructs were then rinsed with 0.1M cacodylate buffer and dehydrated through a graded series of alcohols and critical point dried using a Autosamdri-815 (Tousimis Corp). Then, constructs were mounted on stubs and sputter coated with gold/palladium using a Denton Desk IV (Denton Vacuum). Samples were imaged on a JSM 6610 LV scanning electron microscope at 5 kV (JEOL). FIJI was used to determine density of lumens and distance between lumens.

### Example V: Bead Perfusion Analysis

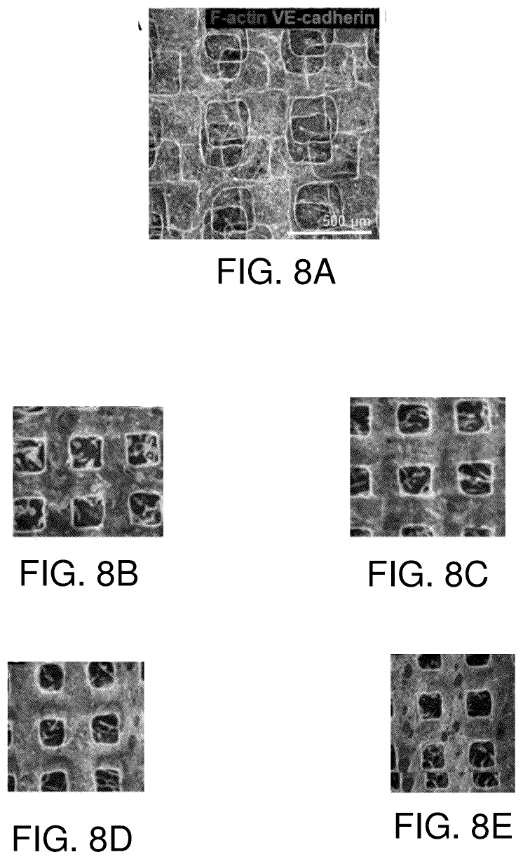

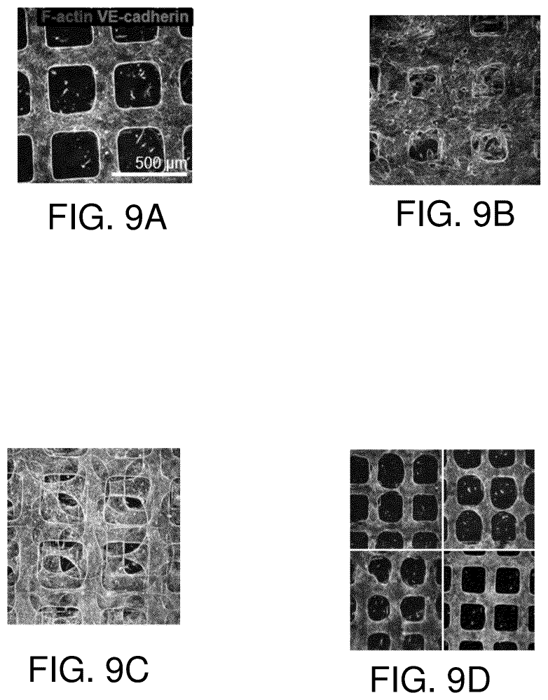

The 3D luminal architecture and perfusability of the multilayer microvessel constructs was analyzed by comparing the 4-layer microvessel constructs (FIGS. 8A-8E) to 1-layer microvessel constructs (FIGS. 9A-9D) with similar conditions. After fixation at day 7 as described above, multilayer and single layer microvessel constructs were attached to a syringe pump and perfused with 1:1000 FluoSpheres Carboxylate-Modified Microspheres beads, 2.0 μm diameter (Thermo Fisher, F8826) diluted in PBS at 10 μL/min. During the perfusion, fluorescent videos were acquired on a Nikon TiE Inverted Widefield Fluorescence Microscope with Yokogawa W1 spinning disk head with no delay. Four videos were acquired for each microvessel construct, with one video for each vessel layer at the center of the focal plane. The field of view for the videos included the 2×2 square section of the grid closest to the inlet, containing the first bifurcation. Bead velocity was quantified in the two straight portions of the pattern closest to the diagonal between the 4th and 5th bifurcations by manual particle tracking on FIJI.

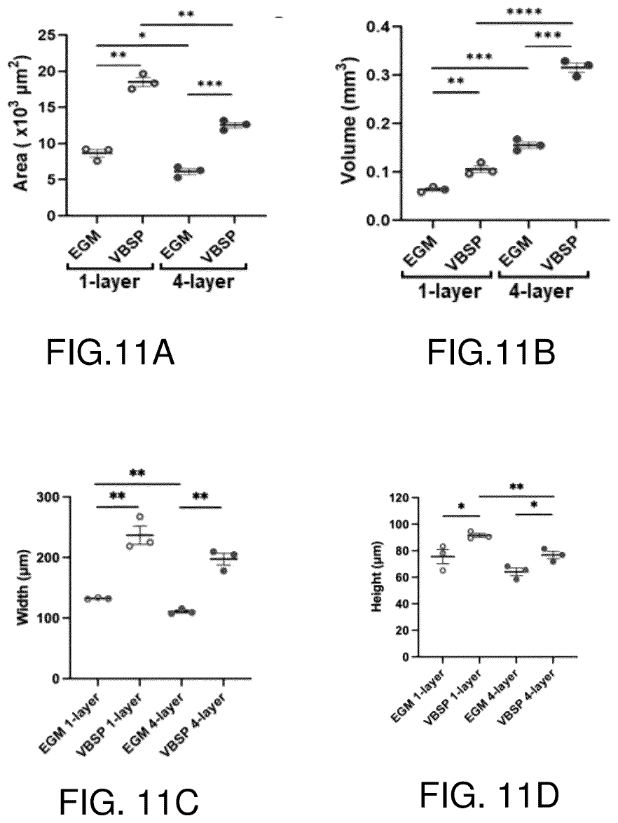

All types of microvessel constructs had lumens with widths greater than 110 μm, the original lithographically defined width, but less height, indicating that vascular patters were compressed during fabrication (FIG. 11C). In both the 1-layer and 4-layer microvessel constructs, culture with vascular basal medium with proangiogenic factors (VBSP) increased the width, height, and subsequently the cross-sectional area of patterned lumen, suggesting vascular re-modeling, as compared to culture conditions with proangiogenic factors (EGM) (FIG. 11A, FIG. 11C, FIG. 11D). Nonetheless, the total luminal volume per field of view was drastically higher in the 4-layer microvessel constructs than the 1-layer microvessel constructs for both the EGM and VBSP conditions, demonstrating that stacking results in higher vascularity (FIG. 11B).

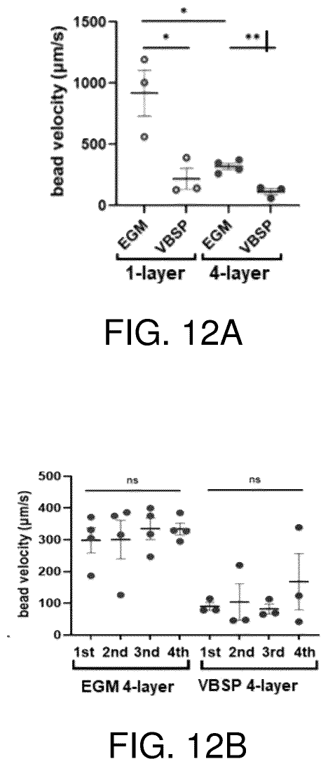

Consistent with the geometry of the constructs, the bead velocity is lower in 4-layer microvessel constructs when compared to 1-layer microvessel constructs and in microvessel constructs with VBSP (with remodeled lumens) when compared to microvessel constructs with EGM-only (EGM 1-layer: 1835.0±375.3; VBSP 1-layer: 434.2±170.7; EGM 4-layer: 633.8±50.7; VBSP 4-layer: 222.3±54.0 mm/s). These trends further confirmed the increased total vessel area in the multilayer constructs, which reduces velocity when flow rate is held constant, and potentially reduces the overall resistance within the construct. When comparing the bead velocity within individual layers of microvessel constructs (FIGS. 12A-12B), there was no statistically significant difference among individual layers for microvessel constructs cultured either with EGM or VBSP, suggesting roughly uniform flow distribution among microvessel layers.

### Example VI: Optical Microangiography Assessment of Perfusable Multilayer Microvessel Constructs

Optical microangiography (OMAG) imaging was used to visualize the 3D perfusable volume within the multilayer microvessel constructs as shown in FIGS. 13A and B. During imaging, microvessel constructs fixed with 4% PF at day 7 were attached to a syringe pump and perfused with 1% Intralipid (Sigma) in PBS at 10 μL/min. A handheld probe-based swept-source OCT (SSOCT) imaging system was used to collect the data. The probe was fixed on a custom-built stand during imaging. The system implemented a vertical-cavity surface-emitting (VCSEL) swept laser source with a central wavelength of 1060 nm and 100 nm spectral bandwidth, giving a ˜10 um axial resolution. The imaging probe was installed with a 5× objective lens with a ˜20 um lateral resolution. The multichannel is set perpendicular to the light beam probe.

Two separate scanning protocols were used to collect structural data for OMAG and to collect velocity data for optical microangiography velocimetry (OMAG-V). In the first protocol for collecting OMAG data, a 9 mm×9 mm field of view was imaged. A B-M mode scanning protocol was performed where 800 A-scans were acquired along the X-direction (fast axis) to compose a single B-scan (cross-section) and 800 B-scans were taken along the Y-direction (slow axis) to generate a 3D volume. Each B-scan was repeated eight times then averaged and log-compressed to reconstruct tissue structure along the Z-direction (depth axis). The en-face images of the OMAG image for the multichannel were generated by maximum intensity projection along the depth.

In the second protocol for OMAG-V data, the data were collected by repeating each B-scan ten times before switching to the following B-scan location until the whole volume was collected. In the fast axis (X-direction scan), each B-scan is composed of 150 A-scans in the Y-direction, and 150 spatial positions were sampled with each position repeatedly scanned for 10 times (i.e. 10 B-scans). A total of 1.6 mm×1.6 mm was covered with this scanning protocol. Temporal changes across the repeated B-scans were used to reconstruct vascular structures and produce velocity data using ED-based OMAG (Gantz, J. A. et al. PLoS One 7, e46971 (2012)). Palpant, N. J. et al. Nat Protoc 12, 15-31 (2017)). A 2D vessel distribution map of the network of the microfluidic channel was generated from the volume scan using the maximum velocity projection along the depth. The averaged velocity from the channel was measured from imaging system was used to collect the data. The probe was fixed on a custom-built stand during imaging. The system implemented a vertical-cavity surface-emitting (VCSEL) swept laser source with a central wavelength of 1060 nm and 100 nm spectral bandwidth, giving a ˜10 um axial resolution. The imaging probe was installed with a 5× objective lens with a ˜20 um lateral resolution. The multichannel is set perpendicular to the light beam probe.

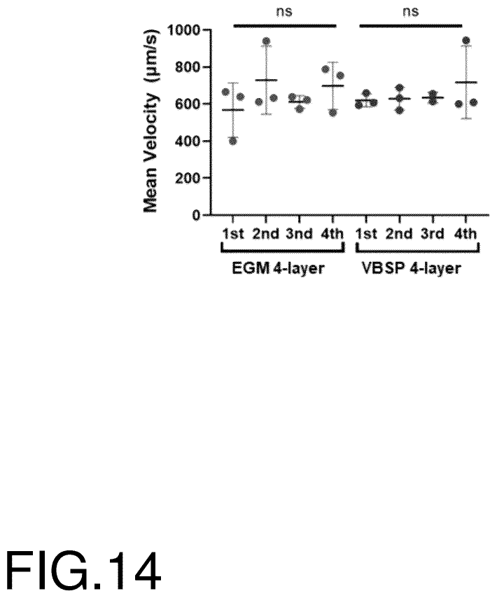

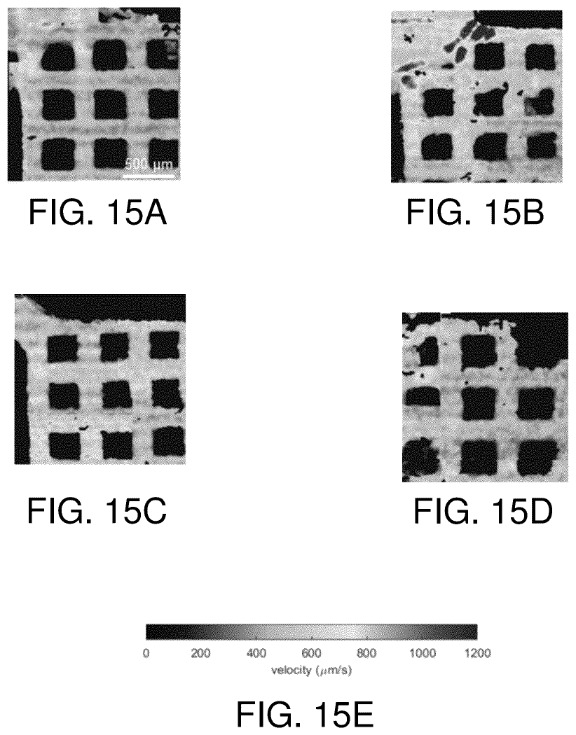

The perfusion dynamics near the inlet of the multilayer microvessel constructs showed similar perfusion profiles throughout each layer, suggesting evenly distributed perfusion as shown in FIGS. 14 and 15A-E. These OMAG-V measurements did not capture a significant difference between EGM and VBSP conditions. While not wishing to be bound, this discrepancy, as compared to bead perfusion, may be due to the larger variation in intralipid particle velocity over the entirety of the vessel branches, as opposed to individual bead velocity measured only in the center of the vessel area. In addition, the projected nature of OMAG-V measurements, through scattering signals, can reduce the sensitivity of velocity measurements. Nonetheless, these results demonstrate the success of building a robust and highly perfusable vasculature with well-distributed flow among vessel layers.

### Example VII: Burst Pressure Testing

To assess the robustness of our multilayer constructs and validate the sealing and adhesion among layers facilitated by the endothelium, burst pressure testing was conducted. To measure the burst pressure of the βV constructs, cellular and acellular 4-layer βVs were connected to a syringe pump and an Elveflow OB1 Mk3 1 psi pressure sensor in parallel. The acquisition frequency for the pressure sensor was set to 60 Hz. Burst pressure was evaluated by doubling the perfusion speed every 5 minutes, starting at 50 μL/min, and assessing if a steady state was reached or if bursting occurred. This experiment was run for 1250 seconds.

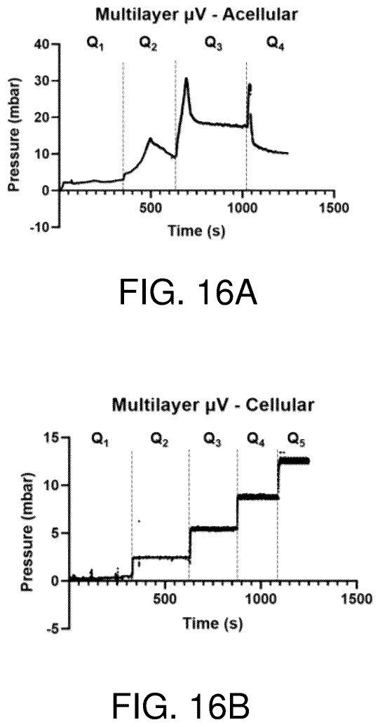

The pressure drop through both acellular and cellular 4-layer βVs when perfused with media at flow rates ranging from 50 μL/min to 800 μL/min was compared as shown in FIGS. 16A and 16B. When the flow rate was at or below 50 μL/min, both acellular and cellular multilayer vessels demonstrated low pressure with minimal fluctuations during the perfusion period. However, as the flow rate increased beyond 50 μL/min, endothelialized multilayer vessels exhibited a distinct step-like increase in pressure following each increment in flow. Conversely, acellular vessels faced challenges in maintaining pressure, and visible separation was observed between the layers throughout the perfusion process in the absence of cells. These findings highlight the importance of endothelialization in enhancing the structural integrity and functionality of integrated multilayer vessels. The presence of cells appears to contribute to the sealing and stability of the multilayer structure, preventing layer separation under increased flow conditions.

### Example VIII: Multilayer Vessels Support Tissue Cell Survival

The capability of the multilayer microvessel constructs in enhancing the tissue cell survival in thick constructs was assessed. As a proof-of-concept, C2C12 mouse myoblasts were used, and their survival was compared between culture within 4-layer microvessel constructs or self-assembled (SA) microvessel constructs after for 7 days. LIVE/DEAD staining of C2C12 vessels was performed after 7 days of culture. A 4 μM solution of both calcein AM and EthD-1 (Thermo Fisher LIVE/DEAD Viability/Cytotoxicity Kit for mammalian cells, L3224) was applied to the inlet and surface of the constructs and incubated at room temperature for 1 hour. Microvessels were then imaged at 10× using a Nikon A1R confocal microscope.

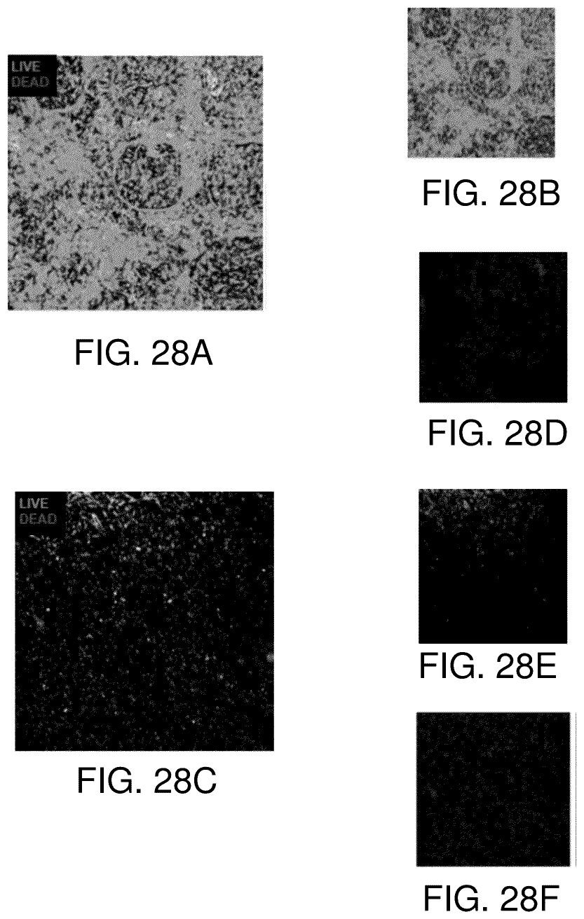

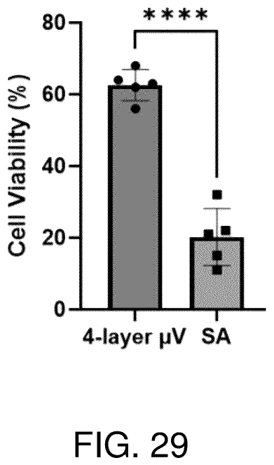

Immunostaining revealed a significantly more robust cellular population within the 4-layer microvessel constructs as compared to the SA control condition (FIGS. 27A and 27B). Additionally, α-actinin staining demonstrated a more relevant C2C12 phenotype specifically within the 4-layer microvessel constructs. LIVE/DEAD staining on both the 4-layer microvessel constructs (FIGS. 28A-C) and the SA constructs (FIGS. 28D-F) revealed significantly greater cell survival within the 4-layer microvessel construct condition (FIG. 29). The presence of perfusable vasculature resulted in approximately a 3-fold increase in cell viability.

### Example IX: Gene Expression of hESC-EC Multilayer Microvessel Constructs

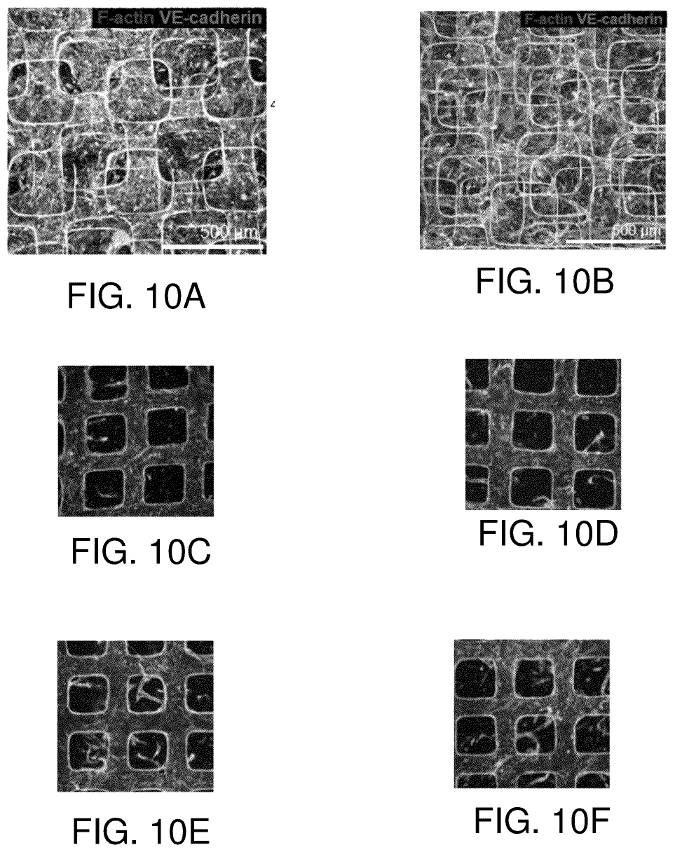

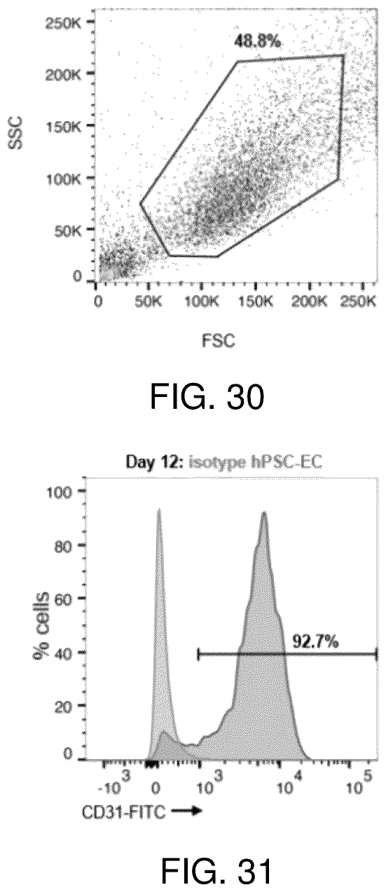

As human stem cell derived endothelial cells (hESC-ECs) would be an ideal cell source for tissue engineering and regenerative medicine applications, 4-layer microvessel constructs with hESC-ECs were generated. Endocardial-like hESC-ECs were differentiated from RUES2 stem cells following Redd, M. A. et al. Nat Commun 10, 584 (2019), and showed >90% purity as indicated by CD31 expression (FIGS. 30-31). Similar to HUVECs, hESC-ECs were successfully seeded and cultured in 4 layered constructs and showed patent lumens after four days of culture (FIG. 10A). Addition of proangiogenic factors (VBSP) also caused hESC-EC multilayer microvessel constructs to undergo significant vascular remodeling and sprouting into the surrounding collagen matrix (FIGS. 10B-F).

### Example X: RNA Seq Analysis

RNAseq analysis was run and transcriptional profiles of hESC-ECs in multilayer microvessel constructs (4-layer βV), single layer microvessel constructs (1-layer βV), and non-perfusable self-assembled constructs (SA) after 4 days of culture were compared.

RNA from hESC-EC multilayer microvessel constructs, hESC-EC single layer microvessel constructs, and hESC-EC self-assembled constructs at day 4 was collected and processed using an RNeasy Micro Kit (Qiagen). RNA quality was assessed with an Agilent High Sensitivity RNA ScreenTape System. All samples had an RNA integrity number greater than 9. RNA samples were prepared using an XT DNA Library Prep Kit (Nextera XT) and a SMART-Seq v4 Ultra Low Input RNA Kit (SMARTv4), a poly-A selection kit using an oligo dT primer. RNA sequencing was performed on an Illumina NovaSeq 6000 SP with paired-end reads. RNAseq data was analyzed using iDEP.95 (integrated, Differential Expression and Pathway analysis, Bioinformatics Research Group, South Dakota State University). Samples were aligned to the hg38 and genes with >1 reads per million in at least a sample were kept for further analysis. edgeR was used for differential expression analysis with genes having fold change >1.5 and FDR<0.05 considered differentially expressed. ShinyGO v0.75 (Gene Ontology Enrichment Analysis, Bioinformatics Research Group, South Dakota State University) was used for GO term analysis and the top 12 relevant terms were displayed.

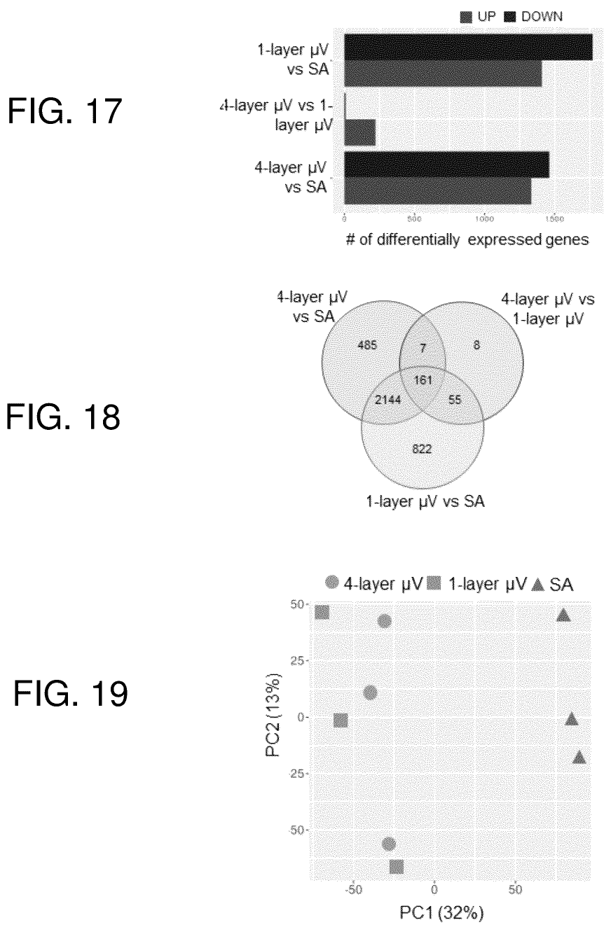

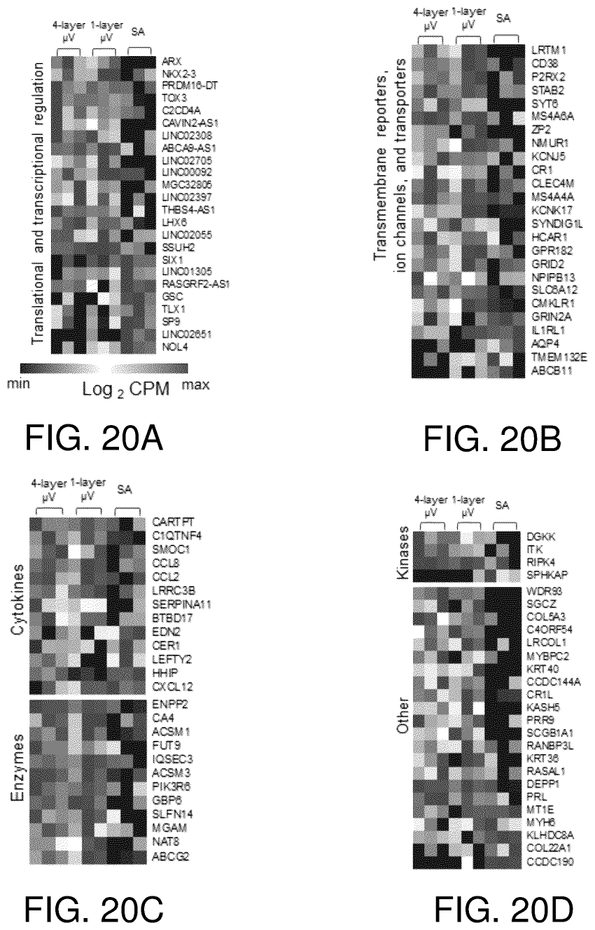

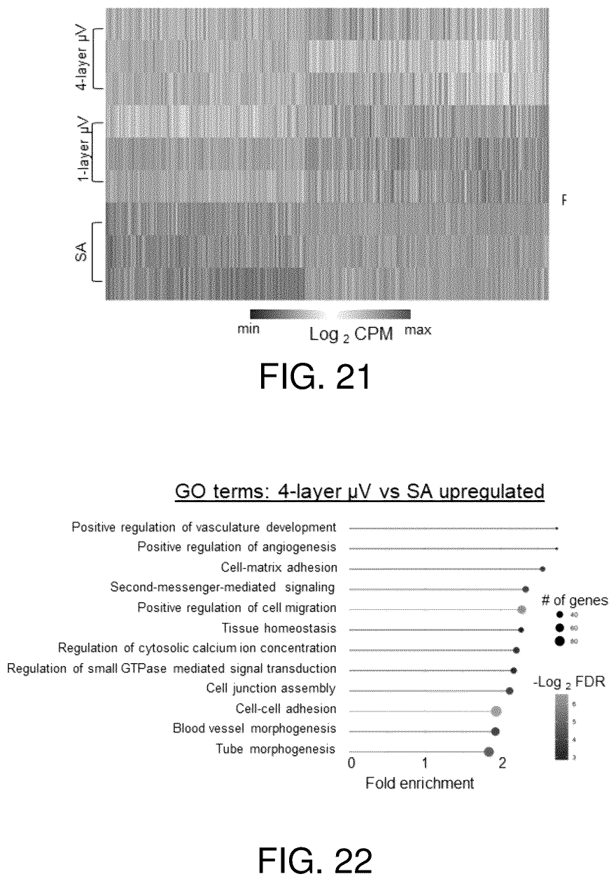

Principal component analysis (PCA) revealed clear separation of the self-assembled constructs from both microvessel conditions, and minor differences between the multilayer and single layer microvessel constructs (FIG. 19). There were 3192 significant differentially expressed genes (fold change >1.5 and FDR<0.05) between single layer microvessel constructs and self-assembled constructs and 2797 differentially expressed genes between multilayer microvessels and self-assembled constructs (FIGS. 17-18). The top differentially expressed genes between multilayer microvessels and self-assembled constructs include translational/transcriptional regulators such as ARX and NKX2-3, transmembrane proteins such as LRTM1 and CD38, cytokines such as CARTPT, and enzymes such as ENPP2 and CA4 (FIGS. 20A-D), with high similarity between single and multilayer microvessel constructs (FIGS. 20-21). Gene Ontology (GO) analysis showed that multilayer microvessel constructs, compared to the self-assembled constructs, were upregulated for positive regulation of angiogenesis, positive regulation of vasculature development, cell-matrix adhesion, second-messenger-mediated signaling, positive regulation of cell migration, and other terms related to vascular remodeling and stability (FIG. 22). Gene expression was much more similar between multilayer and single layer microvessel construct with only 231 differentially expressed genes (FIGS. 17-18). No significant GO terms were found when comparing multilayer and single layer microvessel constructs. These results demonstrate that multilayer microvessel constructs have transcriptional profiles towards vascular development when compared to non-perfusable, self-assembled constructs and that adding layers of vasculature does not significantly change their gene expression.

### Example XI: Rat Surgeries for Ischemia/Reperfusion and Patch Implantation

All animal procedures were approved by the University of Washington Institutional Animal Care and Use Committee (IACUC, protocol #2225-04) and performed in accordance with US NIH Policy on Humane Care and Use of Laboratory Animals, including close monitoring following surgeries.

To determine whether multilayer microvessel constructs would improve host vascular integration in vivo, the implantation and integration of 4-layer microvessel constructs and self-assembled (SA) constructs onto infarcted rat hearts was compared. In this study, 8-week-old, approximately 250-300 g male athymic nude Sprague-Dawley rats were used. All rats had two thoracotomy surgeries that were 4 days apart. For the first surgery, rats underwent an ischemia/reperfusion surgery in which the left descending coronary artery was reversibly ligated for 60 minutes. Prior to the surgery, rats were given an intraperitoneal injection of 68.2 mg/kg ketamine and 4.4 mg/kg xylazine for anesthesia. Rats were also given a second injection of ketamine and xylazine after the ligation began and a subcutaneous injection of sustained release buprenorphine (1 mg/kg) after surgery for analgesia. For the second surgery, collagen patches containing hESC-ECs were implanted onto the surface of the heart over the infarct and attached using two or three 8-0 sutures. The collagen patches were 4-layer microvessel or self-assembled constructs that contained hESC-ECs. They were constructed as described above and cultured for 4 days before being removed from the housing device and plastic shims and cut into a disk (8 mm in diameter×1.5 mm thick) using a biopsy punch. Rats were anesthetized before and during the patch implantation surgery using isoflurane. Again, rats were given sustained release buprenorphine after surgery for analgesia. Additionally, rats received cyclosporine A (5 mg/kg) to prevent cell death by closing the mitochondrial permeability transition pore.

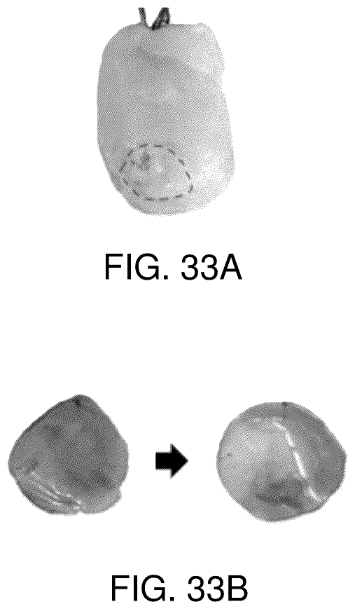

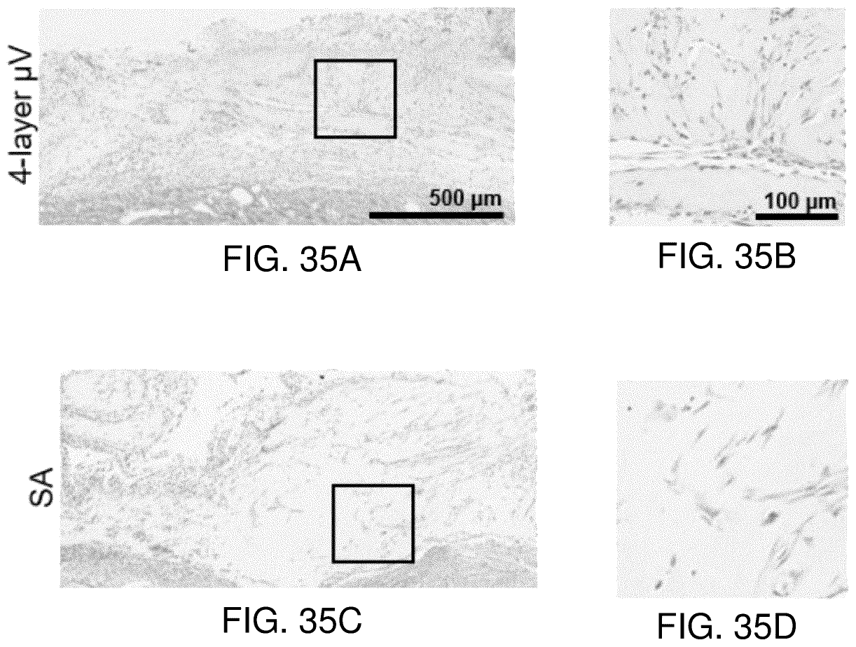

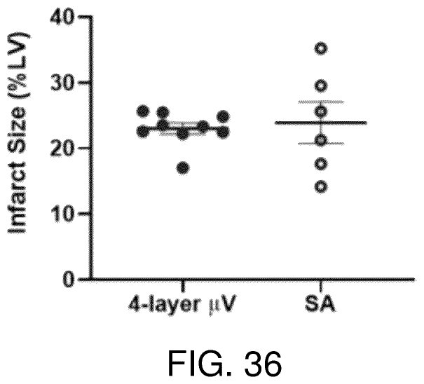

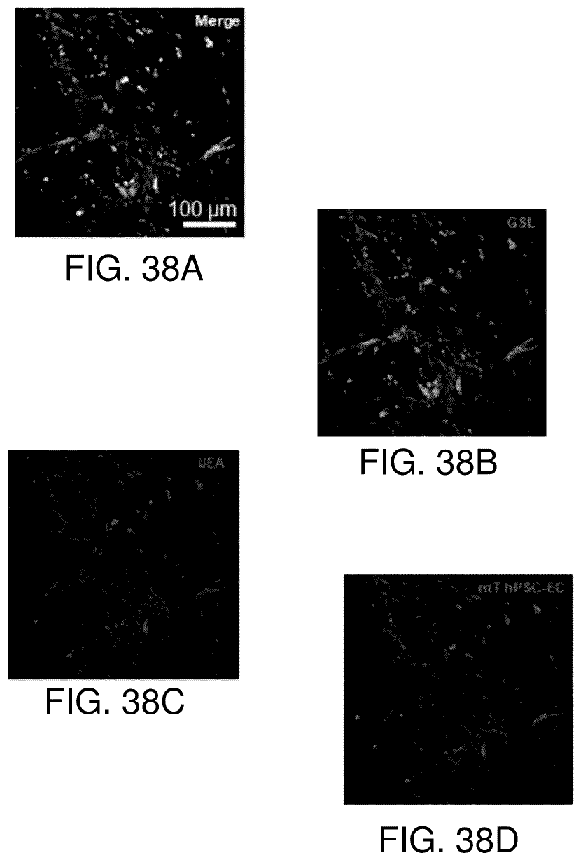

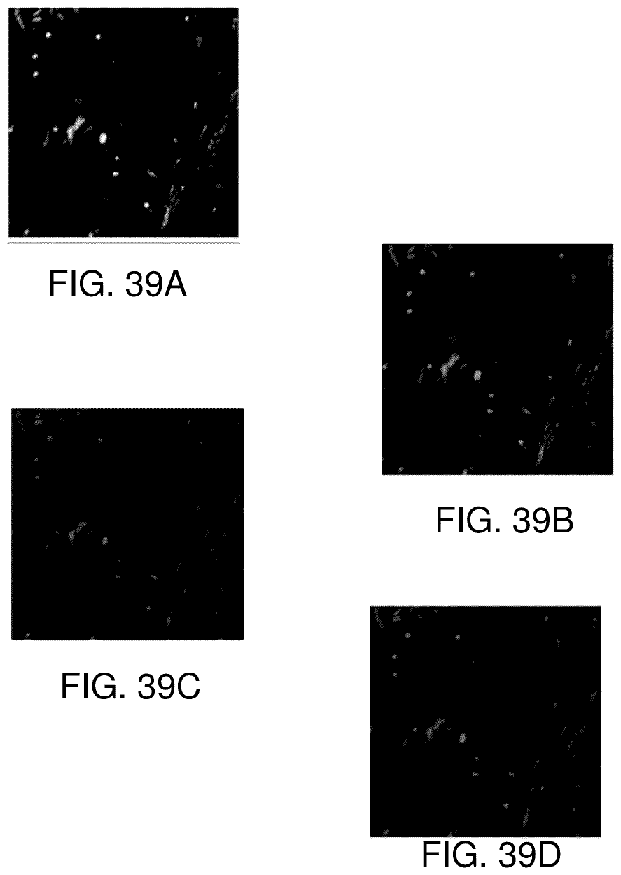

At day 5, SA constructs had formed capillary-sized tubes, but most tubes remained either isolated or connected in very small regions with no perfusion (FIG. 32). Both types of constructs were approximately 1.5 mm thick and were generated 4 days prior to implantation with hESC-ECs expressing m-Tomato (mT) in the bulk collagen matrix (2.5 M/mL) and lining the lumens of the 4-layer microvessel constructs. Constructs were sutured onto the epicardial surface of hearts in rats that had undergone 1-hr ischemiare perfusion of the left anterior descending coronary artery to cause infarction 4 days earlier. Hearts with vascular patches were harvested 5 days after implantation to detect differences in early vascular integration of the graft (FIG. 33B). Upon harvest, hearts were arrested in diastole and fixed via retrograde perfusion of fixative through the coronary vasculature to preserve the vasculature in its most open state. The morphology of the heart and the graft were then assessed via histological analysis of (FIGS. 38A-D) 4-layer βV and (FIGS. 39A-D) SA grafts. Infarct sizes were similar between the 4-layer microvessel group and the self-assembled group (FIGS. 34A, 34B, and 36). Differences in the morphology of the two tissues became apparent with hematoxylin and eosin staining. In multilayer microvessel grafts, many regions of high cellularity could be identified throughout the graft, although the 4-layer grid pattern was no longer identifiable, indicating tissue remodeling (FIGS. 35A-B). In comparison, the self-assembled constructs appeared to have more sparse and singular cellular organization (FIGS. 35C-D).

### Example XII: Tissue Harvest and Retrograde Perfusion Fixation

At 5 days after the ischemia/reperfusion surgery, rats were anesthetized with isoflurane, then given 1.5 mL pentobarbital/phenytoin solution (Euthansol) for euthanasia. After breathing ceased, the chest was opened while the heart was still beating. 50 U Heparin then 3 mL of supersaturated KCl were injected into the inferior vena cava and allowed to circulate. Hearts were then excised, cannulated via the aorta, and attached to a custom-made perfusion system for retrograde perfusion. Vasodilation buffer (PBS with 4 mg/L Papaverine and 1 g/L adenosine) was first perfused through the heart to flush out the blood. Then 4% PF was perfused for 10 minutes at a stable pressure of 100 mmHg to preserve the vasculature. Hearts were additionally fixed at 4% PF overnight before being rinsed with PBS the following day.

To assess the vascular structure and perfusion dynamics within the grafts, a combination of lectin perfusion, tissue clearing, histological analysis, and optical microangiography was used. Prior to sectioning, lectins were perfused through the ex vivo whole hearts via the aorta to label perfusable vasculature that was anastomosed to the host and would have therefore been perfused with blood in vivo. Ulex europaeus lectin was used to identify perfused human vessels, while Griffonia simplicifolia lectin was used to identify all perfused vessels.

After lectin perfusion, hearts were cut into five 2 mm thick sections from the apex. Heart sections were then embedded in paraffin and sliced into 5 μm thick sections and put on slides for histological analysis. Slides were stained for picrosirius red/fast green or hematoxylin and eosin to assess infarct size and gross morphology. Additional slides were stained for immunofluorescent analysis to assess vascularization within the grafts. Briefly, deparaffinized slides were subjected to antigen retrieval by a 25-minute incubation in 10 mM Tris/HCl with 15 μg/mL Proteinase K (Roche) at 37° C. then blocking and permeabilization with 5% NDS and 0.1% Triton-X at room temperature for 1 hour. Next, slides were stained with 1:150 rabbit anti-DsRed (Conetech, 632496), 1:200 goat anti-GSL (Vector, AS-2104), and 1:150 mouse anti-Rhodamine (abcam, ab9093) in 5% NDS overnight, followed by staining with corresponding donkey secondaries. GSL stains both rat and human endothelium while UEA is specific to human endothelium. Because the lectins were perfused through the intact vasculature prior to heart sectioning, GSL stains all perfusable lumens and UEA stains perfusable human lumens. 20× or larger views of the grafts were imaged on a Nikon TiE Inverted Widefield Fluorescence Microscope with Yokogawa W1 spinning disk head. The density and size of GSL+, Rhodamine+(UEA+), DsRed+(mTm hESC-ECs) lumens were quantified using a custom Matlab code and 20× images.

Graft constructs were first punched out using a 6 mm biopsy punch. Samples were washed in PBS for one hour before being blocked overnight in Ce3D alternative blocking buffer (1× PermWash+0.3% TritonX-100+1% bovine serum albumin+1% normal donkey serum in PBS) at 38° C. with slow shaking. Nuclei staining was then performed by the addition of Hoechst (Thermo Fisher, H1399) at a concentration of 1:400 in the Ce3D blocking buffer with the addition of 5% dimethyl sulfoxide and incubated at 38° C. with shaking overnight. Samples were then washed with Ce3D wash buffer (0.3% TritonX-100+0.5% 1-thioglycerol in PBS) for six hours at 38° C. with shaking, with the wash buffer changed every 2 hours. Samples were next incubated in Ce3D clearing solution (40% N-methyl acetamide+Histodenz+0.1% TritonX-100+0.5% 1-thioglycerol), shaking at 38° C. overnight and then moved to room temperature. After a minimum of 24 hours of clearing, z-stacks of the full sample were taken at 10× using a Leica SP8 confocal microscope.

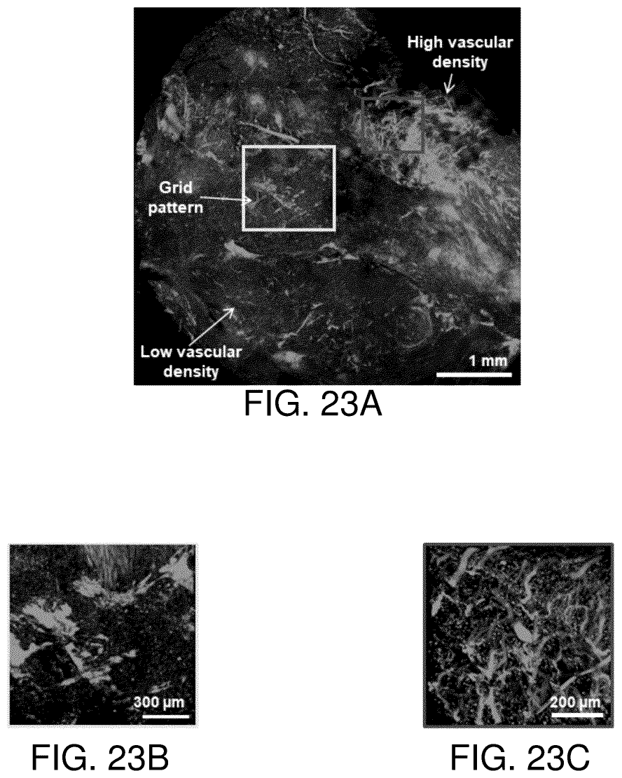

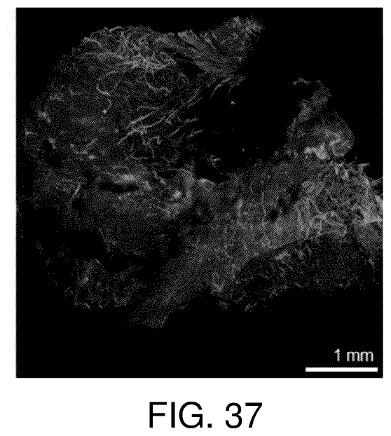

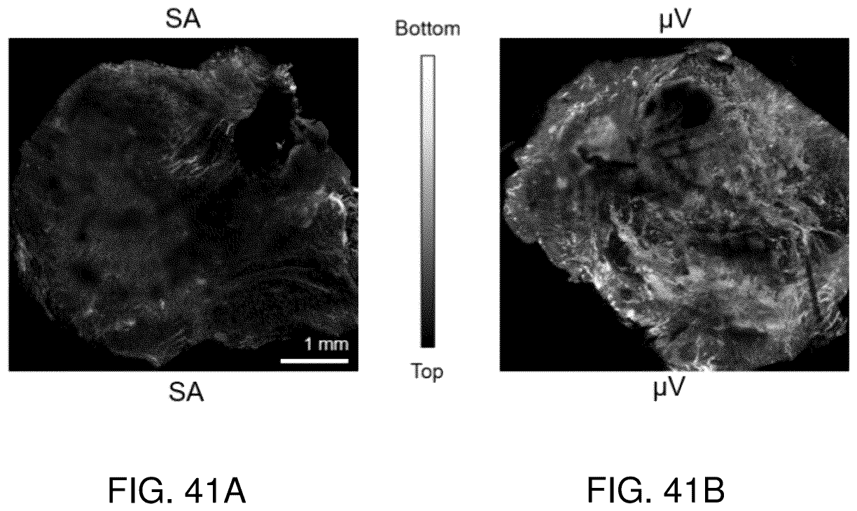

After being biopsy punched and optically cleared, the vascular structure was imaged within whole grafts (FIG. 33B). The multilayer microvessel grafts had significantly greater microvascular density compared to the self-assembled ones (FIGS. 23A-C, FIG. 37). Remodeling in both types of grafts was spatially heterogeneous. In the multilayer microvessel grafts, some small regions maintained the perfusable grid pattern from the initial fabrication, some regions were sparsely vascularized, and in other regions, the pattern was replaced with dense perfusable vasculature (FIGS. 23A-C). Additionally, depth-colored analysis showed that the multilayer microvessel grafts had a greater depth and density of vascularization as compared to the self-assembled grafts (FIGS. 24A-B). Self-assembled grafts contained a very small amount of perfusable vasculature, with most of this vasculature close to the surface of the heart (FIGS. 24A-B, FIG. 41A), while the multilayer microvessel grafts showed dense vasculature throughout, containing both the original grid pattern and highly remodeled structure (FIGS. 24A-B, FIG. 41B).

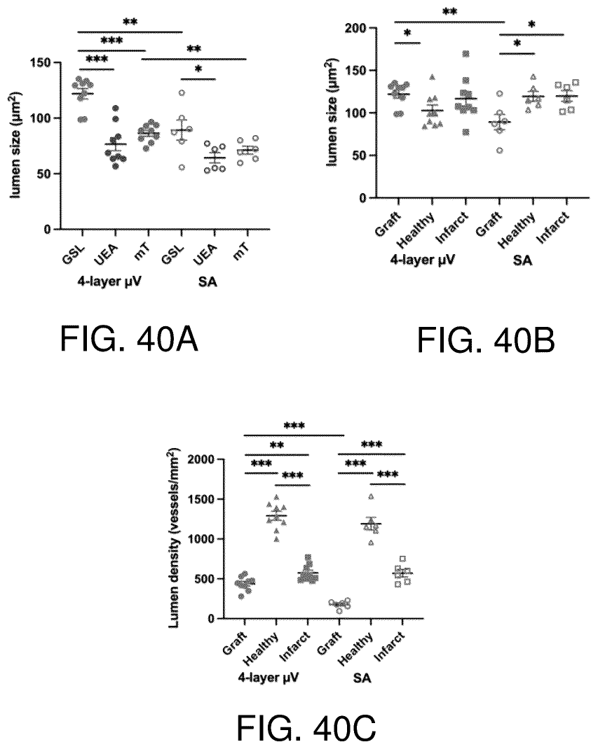

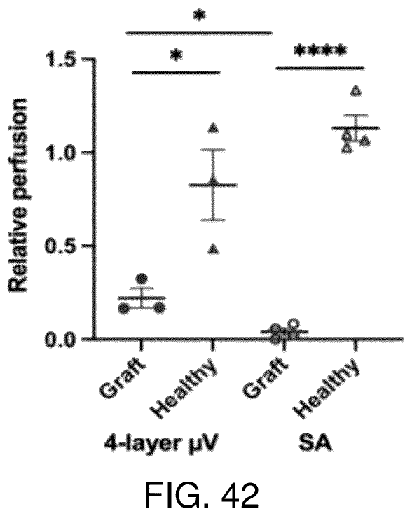

The density of microvessel constructs that were labeled with fluorescein-labeled Griffonia Simplicifolia Lectin (GSL) were quantified for all perfusable vasculature, rhodamine-labeled Ulex Europaeus Agglutinin (UEA) for human-specific perfusable vasculature, and mTomato for human cells that survived implantation (FIG. 41B, FIG. 42). Multilayer microvessel grafts had significantly greater density of both GSL-positive and UEA-positive lumens (GSL+: 437.5±29.2; UEA+: 170.2±15.2 vessels/mm2) when compared to self-assembled grafts (GSL+: 176.3±19.6; UEA+: 64.9±15.6 vessels/mm2), indicating higher vascular integration of multilayer microvessel constructs with the host at early timepoints (FIG. 25A). For both graft conditions, the density and lumen diameter of GSL-positive lumens were significantly greater than the density of UEA-positive lumens, indicating that host vascular ingrowth significantly contributed to the vascularization of the grafts (FIG. 40A). No differences were found in the vascular density or average lumen size in the hearts that are far away from the grafting regions in both grafting conditions (FIG. 40B and FIG. 40C). Multilayer microvessel grafts also had greater density of mTomato-labeled lumens than self-assembled grafts (262.8±25.5; 87.3±14.8 vessels/mm2, respectively), suggesting greater engraftment of implanted cells (FIG. 25A). The perfused vascular density of the grafts is nearly one-third of vessel density in healthy myocardium, but very close (approximately 75%) to that in the underlying infarcted region at this early time point (FIG. 25C).

### Example XIII: Optical Microangiography Assessment of Infarcted Hearts with Microvessel Grafts

Three hearts were randomly selected from each group for OMAG and OMAG-V imaging prior to histological analysis. Hearts were imaged during perfusion of Intralipid at a constant pressure of 100 mmHg using OMAG and OMAG-V protocols described above for multilayer microvessel constructs; however, the field of view was only 4 mm×4 mm for OMAG and 1.6 mm×1.6 mm for OMAG-V. During imaging, each sample was fixed in the center of a Petri dish using a 3D printed mount to prevent sample motion and perfused with 10% Intralipid through the cannulated aorta at a pressure of 100 mmHg. For quantification and visualization, the 3D vascular images were compressed to en-face images using maximum-intensity projection of vessels. Vessel area density was calculated as a percentage of pixels with perfusion signal over the whole imaging field. Relative perfusion was calculated by summing the velocity of every pixel in the image and dividing by the average velocity sum for healthy regions.

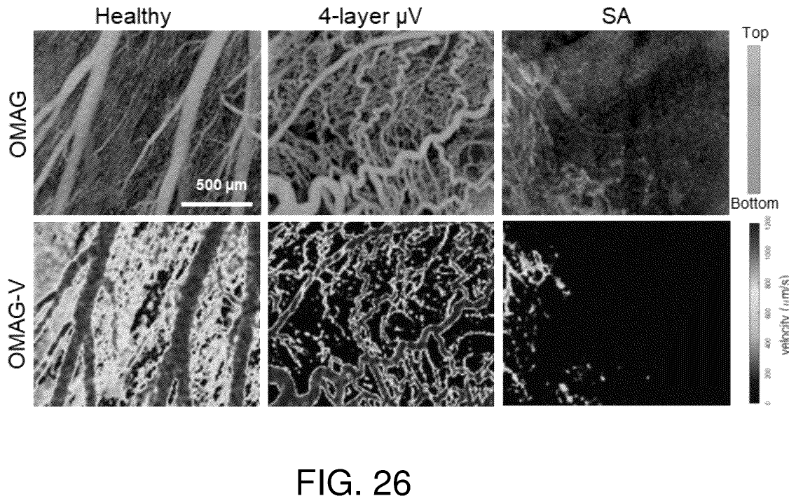

Aligning with the trends from the histological analysis, projected vascular area density was greater in multilayer microvessel grafts (28.1±4.1%) than in self-assembled grafts (6.3±2.4%) though both types of grafts had lower projected vascular area density than healthy regions on the same hearts (4-layer βV: 81.6±11.3; SA: 94.5±3.4%) (FIG. 25B). Notably, the perfusable vasculature of the microvessel grafts appeared to span the depth of the graft, with no obvious layer distinctions (FIG. 26). OMAG-V showed that, while slower than perfusion velocity of vessels in healthy regions, multilayer microvessel grafts had greater average velocity throughout the vasculature than self-assembled grafts (0.28±0.03; 0.12±0.02 mm/s, respectively) (FIG. 25C). Overall perfusion is more than 5-fold greater in the multilayer microvessel grafts (0.22±0.05) than the self-assembled grafts (0.04±0.02) (FIG. 25D, FIG. 42). Taken together, these results suggest that incorporation of perfusable, multilayer microvessel constructs promotes host vascular integration and graft perfusion throughout the full thickness of the tissue.

Statistical analysis: For statistical analysis of two groups, two-tailed t tests assuming unequal variance were used. For analysis of three or more groups, one-way ANOVA was used to test for differences among the groups, followed by pairwise t test with correction for multiple comparisons. All results are presented as mean±standard error of mean. Each data point represents an average for each microvessel or animal and sample numbers represent the number of microvessel constructs or animals analyzed. Significance is represented as * for p<0.05, ** for p<0.01, *** for p<0.001, and **** for p<0.0001.

### Example Clauses

1. A multilayer vascularized construct including:
a plurality of layers of patterned gelled collagen, wherein the patterned gelled collagen includes at least one of endothelial cells, myoblasts, or stromal cells, and
wherein each layer has an inlet and an outlet, and
wherein the layers are integrated into a perfusable integrated construct.

2. The multilayer vascularized construct of clause 1, wherein after fabrication, the multilayer vascularized construct is seeded with cells at a concentration between 2.5 million/mL to 30 million/mL.

3. The multilayer vascularized construct of clause 1 or clause 2, wherein a bottom of a first layer of the plurality of layers and a top of a next layer of the plurality of layers are complementary in shape.

4. The multilayer vascularized construct of clauses 1-3, wherein the construct upregulates positive regulation of angiogenesis in comparison to a self-assembled tissue.

5. The multilayer vascularized construct of clauses 1-3, wherein each layer of the plurality of layers is independently seeded with a same or different types of cells or combinations of types of cells than other layers of the plurality of layers.

6. A method for creating a multilayer vascularized construct, the method including:
preparing a first collagen mixture;
injecting the first collagen mixture into a mold to form a top piece and a bottom piece;
gelling a second collagen mixture between two pieces of elastomeric material to form a collagen membrane; and
sandwiching a plurality of collagen membranes between the top piece and the bottom piece to form a perfusable collagen construct.

7. The method of clause 6, wherein the elastomeric material includes at least one of polydimethylsiloxane, a tri-allyl-tri-azine: tri-thioltriacine 4:3 mixture, tri-allyl-tri-azine: tetra-thiolpentaerythritol 2:3 mixture, photocurable perfluoropolyethers, or cyclicolefin copolymer.

8. The method of clause 6 or clause 7, wherein at least one of the two pieces of elastomeric material has a positive feature.

9. The method of clause 8, wherein the positive feature has a vascular diameter.

10. The method of clause 8 or clause 9, wherein the vascular diameter is 10 μm to 500 μm.

11. The method of clauses 6-10, wherein at least one of the two pieces of elastomeric material has a negative feature.

12. The method of clauses 6-11, further including manufacturing the plurality of collagen membranes in parallel.

13. The method of clauses 6-12, further including culturing the perfusable collagen construct with growth medium.

14. The method of clauses 6-13, wherein the plurality of collagen membranes of the perfusable collagen construct integrate into a cohesive construct.

15. The method of clauses 6-14, further including seeding the second collagen mixture with at least one of endothelial cells, myoblasts, and stromal cells.

16. A method of treating damaged tissue including:
suturing a multilayer vascularized construct over a damaged host tissue,
the multilayer vascularized construct including:
   a plurality of integrated layers of patterned gelled collagen seeded with at least one of endothelial cells, myoblasts, and stromal cells,
wherein a combination of the plurality of integrated layers forms a tertiary structure with perfusable vasculature.

17. The method of clause 16, wherein the plurality of layers includes 4 to 20 layers.

18. The method of clause 16 or 17, wherein the vascularized construct is 2 mm thick.

19. The method of clauses 16-18, wherein the vascularized construct integrates with the host tissue in an area between the vascularized construct and the host tissue.

20. The method of clauses 16-18, wherein the vascularized construct anastomoses to host vasculature after implantation in vivo.

Numerous references have been made to patents, printed publications, journal articles, other written text, and web site content throughout this specification (referenced materials herein). Each of the referenced materials are individually incorporated herein by reference in their entirety for their referenced teaching(s), as of the filing date of the first application in the priority chain in which the specific reference was included. For instance, with regard to chemical compounds, nucleic acid, and amino acids sequences referenced herein that are available in a public database, the information in the database entry is incorporated herein by reference as of the date of an application in the priority chain in which the database identifier for that compound or sequence was first included in the text.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; +18% of the stated value; +17% of the stated value; +16% of the stated value; +15% of the stated value; +14% of the stated value; +13% of the stated value; ±12% of the stated value; +11% of the stated value; +10% of the stated value; ±9% of the stated value; ±8% of the stated value; +7% of the stated value; ±6% of the stated value; ±5% of the stated value; ±4% of the stated value; ±3% of the stated value; +2% of the stated value; or +1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

What is claimed is:

1. A multilayer vascularized construct comprising:
a plurality of layers of patterned gelled collagen, wherein the patterned gelled collagen comprises at least one of endothelial cells, myoblasts, or stromal cells, and
wherein each layer has an inlet and an outlet, and
wherein the layers are integrated into a perfusable integrated construct.

2. The multilayer vascularized construct of claim 1, wherein after fabrication, the multilayer vascularized construct is seeded with cells at a concentration between 2.5 million/mL to 30 million/mL.

3. The multilayer vascularized construct of claim 1, wherein a bottom of a first layer of the plurality of layers and a top of a next layer of the plurality of layers are complementary in shape.

4. The multilayer vascularized construct of claim 1, wherein the construct upregulates positive regulation of angiogenesis in comparison to a self-assembled tissue.

5. The multilayer vascularized construct of claim 1, wherein each layer of the plurality of layers is independently seeded with a same or different types of cells or combinations of types of cells than other layers of the plurality of layers.

6. A method for creating a multilayer vascularized construct, the method comprising:
preparing a first collagen mixture;
injecting the first collagen mixture into a mold to form a top piece and a bottom piece;
gelling a second collagen mixture between two pieces of elastomeric material to form a collagen membrane; and
sandwiching a plurality of collagen membranes between the top piece and the bottom piece to form a perfusable collagen construct.

7. The method of claim 6, wherein the elastomeric material comprises at least one of polydimethylsiloxane, a tri-allyl-tri-azine: tri-thioltriacine 4:3 mixture, tri-allyl-tri-azine: tetra-thiolpentaerythritol 2:3 mixture, photocurable perfluoropolyethers, or cyclicolefin copolymer.

8. The method of claim 7, wherein at least one of the two pieces of elastomeric material has a positive feature.

9. The method of claim 8, wherein the positive feature has a vascular diameter.

10. The method of claim 9, wherein the vascular diameter is 10 μm to 500 μm.

11. The method of claim 7, wherein at least one of the two pieces of elastomeric material has a negative feature.

12. The method of claim 6, further comprising manufacturing the plurality of collagen membranes in parallel.

13. The method of claim 6, further comprising culturing the perfusable collagen construct with growth medium.

14. The method of claim 13, wherein the plurality of collagen membranes of the perfusable collagen construct integrate into a cohesive construct.

15. The method of claim 6, further comprising seeding the second collagen mixture with at least one of endothelial cells, myoblasts, and stromal cells.

16. A method of treating damaged tissue comprising:
suturing a multilayer vascularized construct over a damaged host tissue,
the multilayer vascularized construct comprising:
a plurality of integrated layers of patterned gelled collagen seeded with at least one of endothelial cells, myoblasts, and stromal cells,
wherein a combination of the plurality of integrated layers forms a tertiary structure with perfusable vasculature.

17. The method of claim 16, wherein the plurality of layers comprises 4 to 20 layers.

18. The method of claim 16, wherein the vascularized construct is 2 mm thick.

19. The method of claim 16, wherein the vascularized construct integrates with the host tissue in an area between the vascularized construct and the host tissue.

20. The method of claim 16, wherein the vascularized construct anastomoses to host vasculature after implantation in vivo.
